(19) United States
(12) Patent Application Publication
(10) Pub. No.: US 2020/0253966 A1
(43) Pub. Date: Aug. 13, 2020

(54) CRAC CHANNEL INHIBITOR COMPOSITIONS

(71) Applicant: CalciMedica, Inc., La Jolla, CA (US)

(72) Inventors: Kenneth A. STAUDERMAN, La Jolla, CA (US); Michael DUNN, La Jolla, CA (US)

(21) Appl. No.: 16/481,380

(22) PCT Filed: Jan. 26, 2018
(86) PCT No.: PCT/US18/15555

§ 371 Date: Jul. 26, 2019

Related U.S. Application Data
(60) Provisional application No. 62/451,020, filed on Jan. 26, 2017.

(51) Int. Cl. A61K 31/497; A61K 47/32; A61K 47/28; A61K 47/26; A61K 47/10; A61K 47/14; A61K 9/00; A61K 9/107; A61K 9/19

(52) U.S. Cl. CPC A61K 31/497; A61K 47/32; A61K 47/28; A61K 47/26; A61K 9/19; A61K 47/14; A61K 9/0019; A61K 9/1075; A61K 47/10

(57) ABSTRACT

Provided herein are pharmaceutical compositions, such as emulsions and nanosuspensions, comprising a CRAC channel inhibitor. Also provided herein are methods of treating pancreatitis, viral infections, stroke, traumatic brain injury, fibrosis, inflammation, and autoimmune diseases by administering such pharmaceutical compositions.

### CROSS-REFERENCE

This application is a U.S. National Stage entry of International Application No. PCT/US2018/015555, filed Jan. 26, 2018, which claims the benefit of priority from U.S. Provisional Patent Application No. 62/451,020, filed Jan. 26, 2017, each of which is incorporated by reference in their entirety.

### BACKGROUND

Calcium plays a vital role in cell function and survival. For example, calcium is a key element in the transduction of signals into and within cells. Cellular responses to growth factors, neurotransmitters, hormones, and a variety of other signal molecules are initiated through calcium-dependent processes.

Virtually all cell types depend in some manner upon the generation of cytoplasmic Ca²⁺ signals to regulate cell function, or to trigger specific responses. Cytosolic Ca²⁺ signals control a wide array of cellular functions ranging from short-term responses, such as contraction and secretion, to longer-term regulation of cell growth and proliferation. Usually, these signals involve some combination of release of Ca²⁺ from intracellular stores, such as the endoplasmic reticulum (ER), and influx of Ca²⁺ across the plasma membrane. In one example, cell activation begins with an agonist binding to a surface membrane receptor, which is coupled to phospholipase C (PLC) through a G-protein mechanism. PLC activation leads to the production of inositol 1,4,5-triphosphate (IP3), which in turn activates the IP3 receptor causing release of Ca²⁺ from the ER. The fall in ER Ca²⁺ then signals to activate plasma membrane store-operated calcium (SOC) channels.

Store-operated calcium (SOC) influx is a process in cellular physiology that controls such diverse functions such as, but not limited to, refilling of intracellular Ca²⁺ stores (Putney et al. Cell, 75, 199-201, 1993), activation of enzymatic activity (Fagan et al., J. Biol. Chem. 275:26530-26537, 2000), gene transcription (Lewis, Annu. Rev. Immunol. 19:497-521, 2001), cell proliferation (Nunez et al., J Physiol. 571.1, 57-73, 2006), and release of cytokines (Winslow et al., Curr. Opin. Immunol. 15:299-307, 2003). In some nonexcitable cells, e.g., blood cells, immune cells, hematopoietic cells, T lymphocytes, and mast cells, SOC influx occurs through calcium release-activated calcium (CRAC) channels, a type of SOC channel.

### SUMMARY OF THE INVENTION

Provided herein are embodiments related to pharmaceutical compositions comprising a CRAC Channel inhibitor and methods of treating pancreatitis, viral infections, stroke, traumatic brain injury, fibrosis, inflammation, and autoimmune diseases in a mammal such as a person using such pharmaceutical compositions.

Disclosed herein is a pharmaceutical composition comprising N-(5-(6-chloro-2,2-difluorobenzo[d][1,3]dioxol-5-yl)pyrazin-2-yl)-2-fluoro-6-methylbenzamide, or a pharmaceutically acceptable salt thereof and a pharmaceutically acceptable excipient. In some embodiments, the pharmaceutical composition is formulated as a homogeneous liquid, an emulsion, a nanosuspension, or a powder for reconstitution. In some embodiments, the pharmaceutical composition is suitable for injection. In some embodiments, N-(5-(6-chloro-2,2-difluorobenzo[d][1,3]dioxol-5-yl)pyrazin-2-yl)-2-fluoro-6-methylbenzamide is present as a free base. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof is crystalline. In some embodiments, crystalline N-(5-(6-chloro-2,2-difluorobenzo[d][1,3]dioxol-5-yl)pyrazin-2-yl)-2-fluoro-6-methylbenzamide is crystalline Form A which has at least one of the following properties: (a) an X-Ray powder diffraction (XRPD) pattern substantially the same as shown in FIG. 1; (b) an X-ray powder diffraction (XRPD) pattern comprising characteristic peaks at about 13.8° 2-Theta, about 14.2° 2-Theta, about 16.8° 2-Theta, about 19.2° 2-Theta, about 19.7° 2-Theta, about 21.1° 2-Theta, about 22.5° 2-Theta, about 22.7° 2-Theta, about 26.5° 2-Theta, and about 27.5° 2-Theta; (c) a DSC thermogram substantially similar to the one set forth in FIG. 2; or (d) a DSC thermogram with an endotherm having a peak at about 156.6° C. In some embodiments, the pharmaceutical composition is formulated as an emulsion. In some embodiments, the emulsion is suitable for injection. In some embodiments, the pharmaceutically acceptable excipient is selected from the group consisting of lecithin, soybean oil (SBO), Medium Chain Triglycerides (MCT), cholesterol, Vitamin E succinate (VES), sucrose, glycerin, EDTA-Na₂, and any combination thereof. In some embodiments the pharmaceutical composition comprises: (i)N-(5-(6-chloro-2,2-difluorobenzo[d][1,3]dioxol-5-yl)pyrazin-2-yl)-2-fluoro-6-methylbenzamide; (ii) lecithin; (iii) Medium Chain Triglycerides (MCT); (iv) Glycerin; and (v) Water. In some embodiments, the N-(5-(6-chloro-2,2-difluorobenzo[d][1,3]dioxol-5-yl)pyrazin-2-yl)-2-fluoro-6-methylbenzamide is present at a concentration from about 0.1 mg/mL to about 4.0 mg/mL. In some embodiments, the N-(5-(6-chloro-2,2-difluorobenzo[d][1,3]dioxol-5-yl)pyrazin-2-yl)-2-fluoro-6-methylbenzamide is present at a concentration of less than about 1.8 mg/mL. In some embodiments, the N-(5-(6-chloro-2,2-difluorobenzo[d][1,3]dioxol-5-yl)pyrazin-2-yl)-2-fluoro-6-methylbenzamide is present at a concentration of about 1.6 mg/mL. In some embodiments, the N-(5-(6-chloro-2,2-difluorobenzo[d][1,3]dioxol-5-yl)pyrazin-2-yl)-2-fluoro-6-methylbenzamide is present at a concentration from about 0.1% to about 1% (w/w). In some embodiments, the N-(5-(6-chloro-2,2-difluorobenzo[d][1,3]dioxol-5-yl)pyrazin-2-yl)-2-fluoro-6-methylbenzamide is present at a concentration from about 0.1% to about 0.3% (w/w). In some embodiments, the lecithin is egg lecithin. In some embodiments, the lecithin is present at a concentration from about 5% to about 15% (w/w). In some embodiments, the lecithin is present at a concentration of about 10% (w/w).

In some embodiments, the Medium Chain Triglycerides (MCT) is present at a concentration from about 1% to about 10% (w/w). In some embodiments, the Medium Chain Triglycerides (MCT) is present at a concentration of about 5% (w/w). In some embodiments, the Glycerin is present at a concentration from about 1% to about 5% (w/w). In some embodiments, the Glycerin is present at a concentration of about 2.25% (w/w). In some embodiments the pharmaceutical composition further comprises EDTA-Na₂. In some embodiments, the EDTA-Na₂ is present at a concentration from about 0.001% to about 0.01% (w/w). In some embodiments, the EDTA-Na₂ is present at a concentration of about 0.005%. In some embodiments the pharmaceutical composition has a pH from about 4 to about 9. In some embodiments the pharmaceutical composition has a pH from about 6 to about 8. In some embodiments the pharmaceutical composition has a pH of about 7. In some embodiments, the pH is adjusted by addition of HCl or NaOH. In some embodiments the pharmaceutical composition is substantially free of N-(5-(6-chloro-2,2-difluorobenzo[d][1,3]dioxol-5-yl)pyrazin-2-yl)-2-fluoro-6-methylbenzamide crystalline Form B which has at least one of the following properties: (a) an X-Ray powder diffraction (XRPD) pattern substantially the same as shown in FIG. 4; (b) an X-ray powder diffraction (XRPD) pattern comprising characteristic peaks at about 14.2° 2-Theta, about 17.1° 2-Theta, about 21.5° 2-Theta, about 25.4° 2-Theta, about 26.5° 2-Theta, and about 26.9° 2-Theta; (c) a DSC thermogram substantially similar to the one set forth in FIG. 5; or (d) a DSC thermogram with an endotherm having a peak at about 54.3° C. and about 155.9° C. In some embodiments, the pharmaceutical composition is stable at about 5±3° C. for at least 3 months. In some embodiments, the pharmaceutical composition is stable at about 5±3° C. for at least 6 months. In some embodiments, the pharmaceutical composition is stable at about 5±3° C. for at least 12 months. In some embodiments, the pharmaceutical composition is stable at about 25±3° C. for at least 3 months. In some embodiments, the pharmaceutical composition is stable at about 25±3° C. for at least 6 months. In some embodiments, the pharmaceutical composition is stable at about 25±3° C. for at least 12 months. In some embodiments, the pharmaceutical composition is formulated as a powder for reconstitution. In some embodiments, the pharmaceutical composition is suitable for injection once reconstituted with an aqueous carrier. In some embodiments, the aqueous carrier is selected from the group consisting of water, saline, 5% dextrose in water, 5% dextrose in saline, and any combination thereof. In some embodiments, the pharmaceutical composition is in the form of a nanosuspension once reconstituted. In some embodiments, the nanosuspension comprises nanoparticles. In some embodiments, each nanoparticle has an average diameter from about 50 nm to about 500 nm. In some embodiments, each nanoparticle has an average diameter from about 50 nm to about 150 nm. In some embodiments, each nanoparticle has an average diameter of about 100 nm. In some embodiments, the pharmaceutically acceptable excipient is selected from the group consisting of polyvinylpyrrolidone (PVP), sodium deoxycholate, and any combination thereof. In some embodiments the pharmaceutical composition further comprises a cryoprotectant. In some embodiments, the cryoprotectant is selected the group consisting of from sucrose, sucrose/mannitol, trehalose, trehalose/mannitol, and any combination thereof. In some embodiments the pharmaceutical composition comprises: (i)N-(5-(6-chloro-2,2-difluorobenzo[d][1,3]dioxol-5-yl)pyrazin-2-yl)-2-fluoro-6-methylbenzamide; (ii) polyvinylpyrrolidone (PVP); (iii) sodium deoxycholate; and (iv) sucrose. In some embodiments, the N-(5-(6-chloro-2,2-difluorobenzo[d][1,3]dioxol-5-yl)pyrazin-2-yl)-2-fluoro-6-methylbenzamide is present at a concentration from about 1 mg/mL to about 100 mg/mL, once reconstituted. In some embodiments, the N-(5-(6-chloro-2,2-difluorobenzo[d][1,3]dioxol-5-yl)pyrazin-2-yl)-2-fluoro-6-methylbenzamide is present at a concentration of about 50 mg/mL, once reconstituted. In some embodiments, the polyvinylpyrrolidone (PVP) is present at a concentration from about 0.1% to about 5% (w/w). In some embodiments, the polyvinylpyrrolidone (PVP) is present at a concentration of about 0.5% (w/w). In some embodiments, the sodium deoxycholate is present at a concentration from about 0.1% to about 1% (w/w). In some embodiments, the sodium deoxycholate is present at a concentration of about 0.125% (w/w). In some embodiments, the sucrose is present at a concentration from about 1% to about 20% (w/w). In some embodiments, the sucrose is present at a concentration of about 10% (w/w). In some embodiments the pharmaceutical composition has a pH from about 4 to about 9 once reconstituted. In some embodiments the pharmaceutical composition has a pH of about 7 once reconstituted. In some embodiments, the pharmaceutical composition is stable at about 5±3° C. for at least 3 months once reconstituted. In some embodiments, the pharmaceutical composition is stable at about 5±3° C. for at least 6 months once reconstituted. In some embodiments, the pharmaceutical composition is stable at about 5±3° C. for at least 12 months once reconstituted. In some embodiments, the pharmaceutical composition is stable at about 25±3° C. for at least 3 months once reconstituted. In some embodiments, the pharmaceutical composition is stable at about 25±3° C. for at least 6 months once reconstituted. In some embodiments, the pharmaceutical composition is stable at about 25±3° C. for at least 12 months once reconstituted.

Also disclosed herein are methods of treating pancreatitis in an individual in need thereof comprising administering to the individual a pharmaceutical composition disclosed herein. Also disclosed herein are methods of treating idiopathic pulmonary fibrosis (IPF) in an individual in need thereof comprising administering to the individual a pharmaceutical composition disclosed herein. Also disclosed herein are methods of treating stroke or traumatic brain injury in an individual in need thereof comprising administering to the individual a pharmaceutical composition disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the XRPD pattern of N-(5-(6-chloro-2,2-difluorobenzo[d][1,3]dioxol-5-yl)pyrazin-2-yl)-2-fluoro-6-methylbenzamide Form A.

FIG. 2 shows the TGA and DSC curves of N-(5-(6-chloro-2,2-difluorobenzo[d][1,3]dioxol-5-yl)pyrazin-2-yl)-2-fluoro-6-methylbenzamide Form A.

FIG. 3 shows the DVS of N-(5-(6-chloro-2,2-difluorobenzo[d][1,3]dioxol-5-yl)pyrazin-2-yl)-2-fluoro-6-methylbenzamide Form A.

FIG. 4 shows the XRPD pattern of N-(5-(6-chloro-2,2-difluorobenzo[d][1,3]dioxol-5-yl)pyrazin-2-yl)-2-fluoro-6-methylbenzamide Form B as compared to Form A.

FIG. 5 shows the DSC curve of N-(5-(6-chloro-2,2-difluorobenzo[d][1,3]dioxol-5-yl)pyrazin-2-yl)-2-fluoro-6-methylbenzamide Form B as compared to Form A.

FIG. 6 shows the XRPD pattern of N-(5-(6-chloro-2,2-difluorobenzo[d][1,3]dioxol-5-yl)pyrazin-2-yl)-2-fluoro-6-methylbenzamide Form C as compared to Form A

FIG. 7 shows the DSC curve of N-(5-(6-chloro-2,2-difluorobenzo[d][1,3]dioxol-5-yl)pyrazin-2-yl)-2-fluoro-6-methylbenzamide Form C as compared to Form A.

FIG. 8 shows the XRPD pattern of N-(5-(6-chloro-2,2-difluorobenzo[d][1,3]dioxol-5-yl)pyrazin-2-yl)-2-fluoro-6-methylbenzamide Form D as compared to Form A

FIG. 9 shows the DSC curve of N-(5-(6-chloro-2,2-difluorobenzo[d][1,3]dioxol-5-yl)pyrazin-2-yl)-2-fluoro-6-methylbenzamide Form D as compared to Form A.

FIG. 10 shows the manufacturing process flowchart for the manufacture of a N-(5-(6-chloro-2,2-difluorobenzo[d][1,3]dioxol-5-yl)pyrazin-2-yl)-2-fluoro-6-methylbenzamide emulsion.

### DETAILED DESCRIPTION OF THE INVENTION

Disclosed herein are pharmaceutical compositions comprising a CRAC channel inhibitor and a pharmaceutically acceptable excipient. In some embodiments, the pharmaceutical composition is formulated as a homogeneous liquid, an emulsion, a nanosuspension, or a powder for reconstitution. In some embodiments, the pharmaceutical composition is formulated as an emulsion. In some embodiments, the pharmaceutical composition is formulated as a nanosuspension. In some embodiments, the pharmaceutical composition is formulated as a powder for reconstitution. In some embodiments, the powder for reconstitution is reconstituted with an aqueous carrier to form a nanosuspension. In some embodiments, the CRAC channel inhibitor is Compound A having the structure

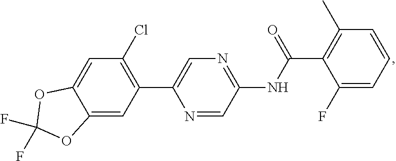 [SMILES: Cc1cccc(F)c1C(=O)Nc1cnc(-c2cc3c(cc2Cl)OC(F)(F)O3)cn1]

or a pharmaceutically acceptable salt thereof. In some embodiments the CRAC channel inhibitor is N-(5-(6-chloro-2,2-difluorobenzo[d][1,3]dioxol-5-yl)pyrazin-2-yl)-2-fluoro-6-methylbenzamide, or a pharmaceutically acceptable salt thereof. In some embodiments, the CRAC channel inhibitor is N-(5-(6-chloro-2,2-difluorobenzo[d][1,3]dioxol-5-yl)pyrazin-2-yl)-2-fluoro-6-methylbenzamide free base. In some embodiments, the CRAC channel inhibitor is crystalline N-(5-(6-chloro-2,2-difluorobenzo[d][1,3]dioxol-5-yl)pyrazin-2-yl)-2-fluoro-6-methylbenzamide, or a pharmaceutically acceptable salt thereof. In some embodiments, the CRAC channel inhibitor is crystalline N-(5-(6-chloro-2,2-difluorobenzo[d][1,3]dioxol-5-yl)pyrazin-2-yl)-2-fluoro-6-methylbenzamide free base.

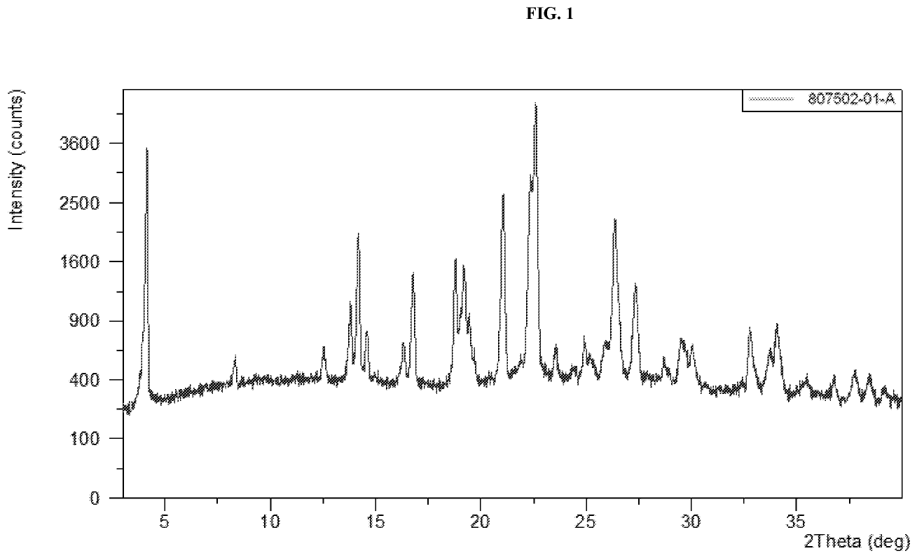

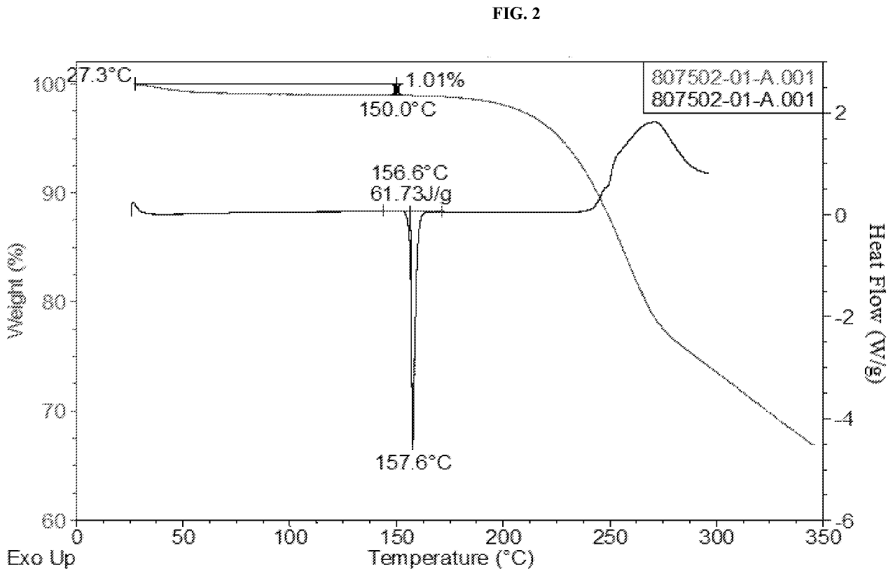

Described herein are pharmaceutical compositions comprising crystalline N-(5-(6-chloro-2,2-difluorobenzo[d][1,3]dioxol-5-yl)pyrazin-2-yl)-2-fluoro-6-methylbenzamide free base Form A which has at least one of the following properties:
(a) an X-Ray powder diffraction (XRPD) pattern substantially the same as shown in FIG. 1;
(b) an X-ray powder diffraction (XRPD) pattern comprising characteristic peaks at about 13.8° 2-Theta, about 14.2° 2-Theta, about 16.8° 2-Theta, about 19.2° 2-Theta, about 19.7° 2-Theta, about 21.1° 2-Theta, about 22.5° 2-Theta, about 22.7° 2-Theta, about 26.5° 2-Theta, and about 27.5° 2-Theta;
(c) a DSC thermogram substantially similar to the one set forth in FIG. 2; or
(d) a DSC thermogram with an endotherm having a peak at about 156.6° C.

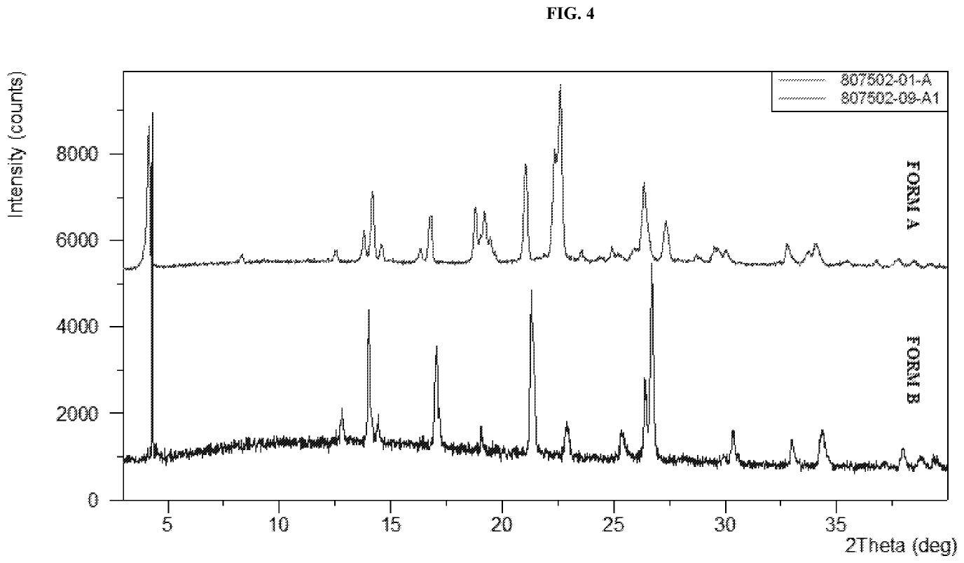

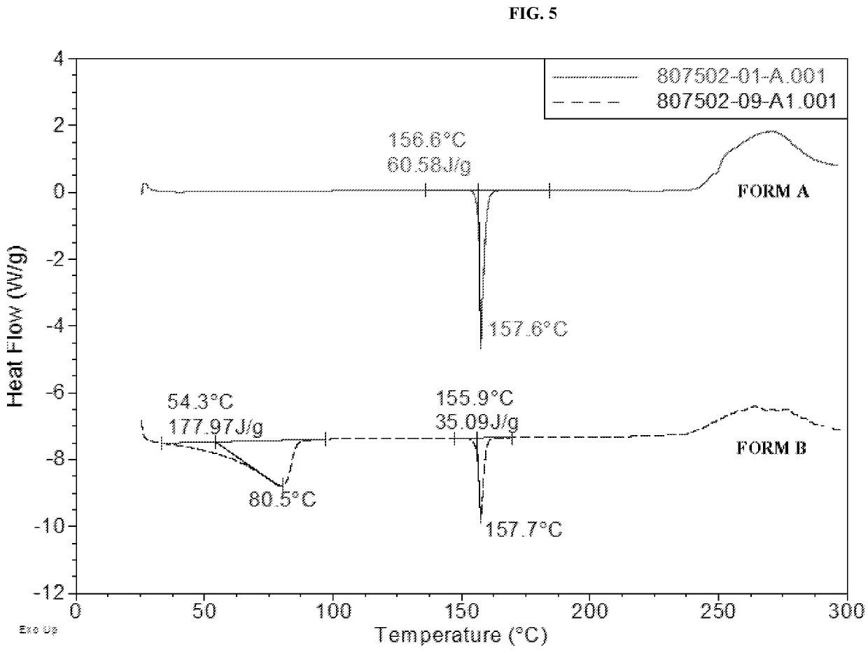

Described herein are pharmaceutical compositions comprising crystalline N-(5-(6-chloro-2,2-difluorobenzo[d][1,3]dioxol-5-yl)pyrazin-2-yl)-2-fluoro-6-methylbenzamide free base Form B which has at least one of the following properties:
(a) an X-Ray powder diffraction (XRPD) pattern substantially the same as shown in FIG. 4;
(b) an X-ray powder diffraction (XRPD) pattern comprising characteristic peaks at about 14.2° 2-Theta, about 17.1° 2-Theta, about 21.5° 2-Theta, about 25.4° 2-Theta, about 26.5° 2-Theta, and about 26.9° 2-Theta;
(c) a DSC thermogram substantially similar to the one set forth in FIG. 5; or
(d) a DSC thermogram with an endotherm having a peak at about 54.3° C. and about 155.9° C.

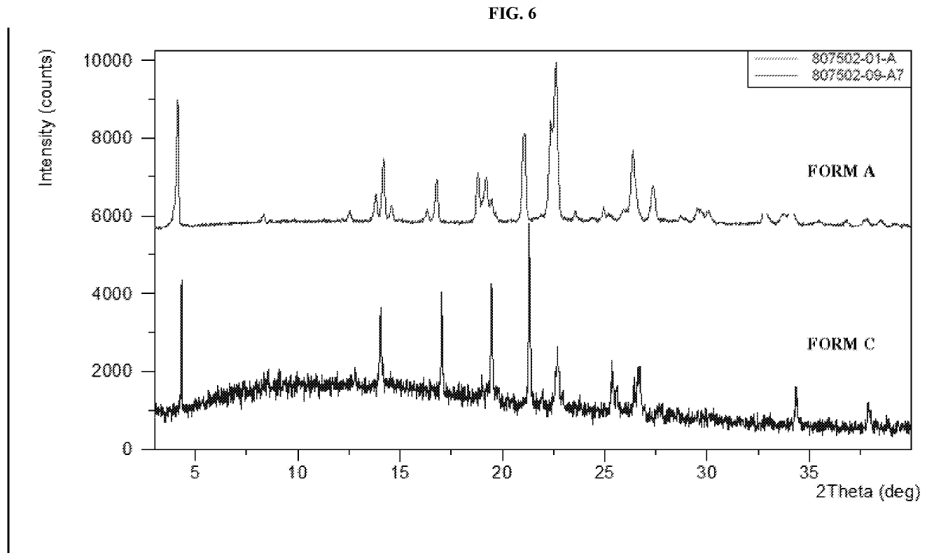

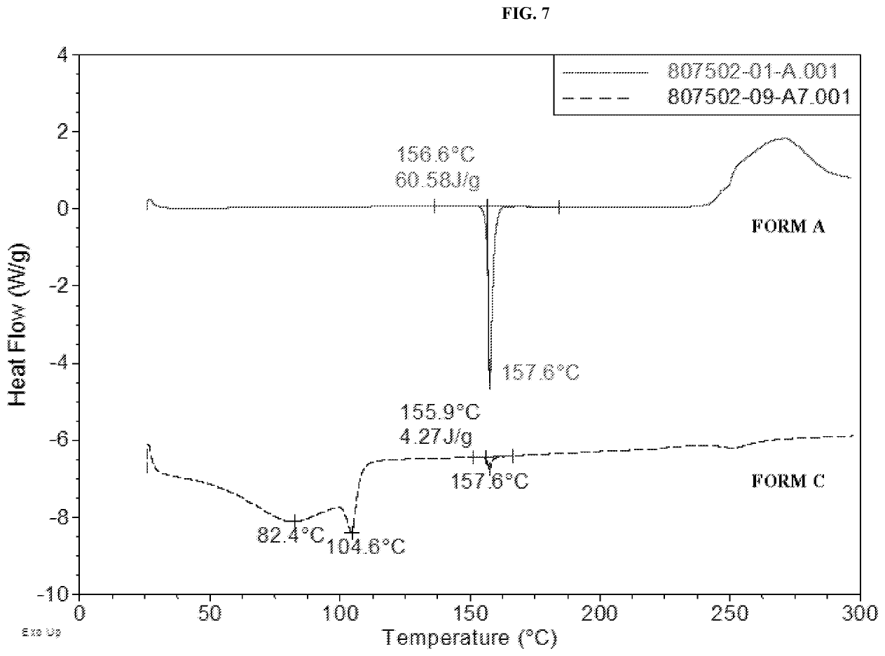

Described herein are pharmaceutical compositions comprising crystalline N-(5-(6-chloro-2,2-difluorobenzo[d][1,3]dioxol-5-yl)pyrazin-2-yl)-2-fluoro-6-methylbenzamide free base Form C which has at least one of the following properties:
(a) an X-Ray powder diffraction (XRPD) pattern substantially the same as shown in FIG. 6;
(b) an X-ray powder diffraction (XRPD) pattern comprising characteristic peaks at about 14.1° 2-Theta, about 17.1° 2-Theta, about 19.6° 2-Theta, about 21.4° 2-Theta, about 22.5° 2-Theta, about 25.4° 2-Theta, about 25.9° 2-Theta, and about 34.3° 2-Theta;
(c) a DSC thermogram substantially similar to the one set forth in FIG. 7; or
(d) a DSC thermogram with an endotherm having a peak at about 82.4° C. and about 104.6° C.

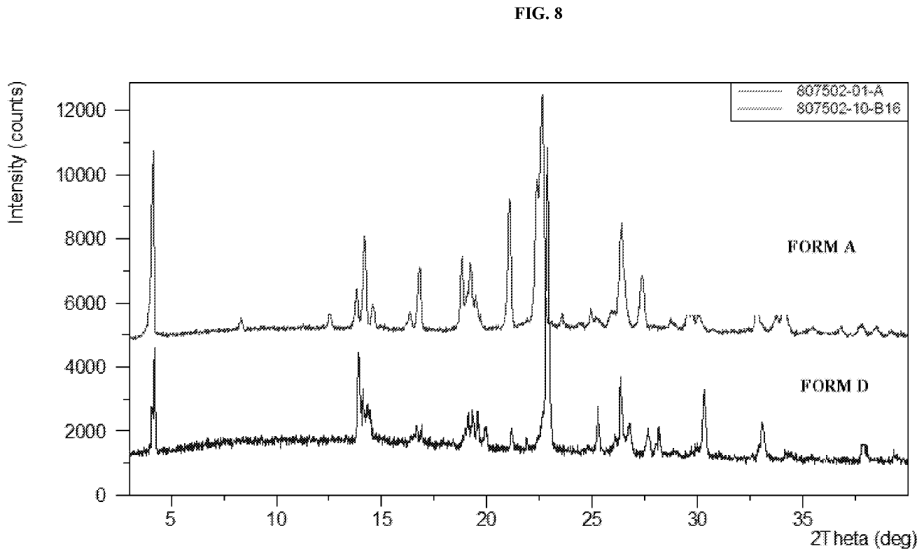

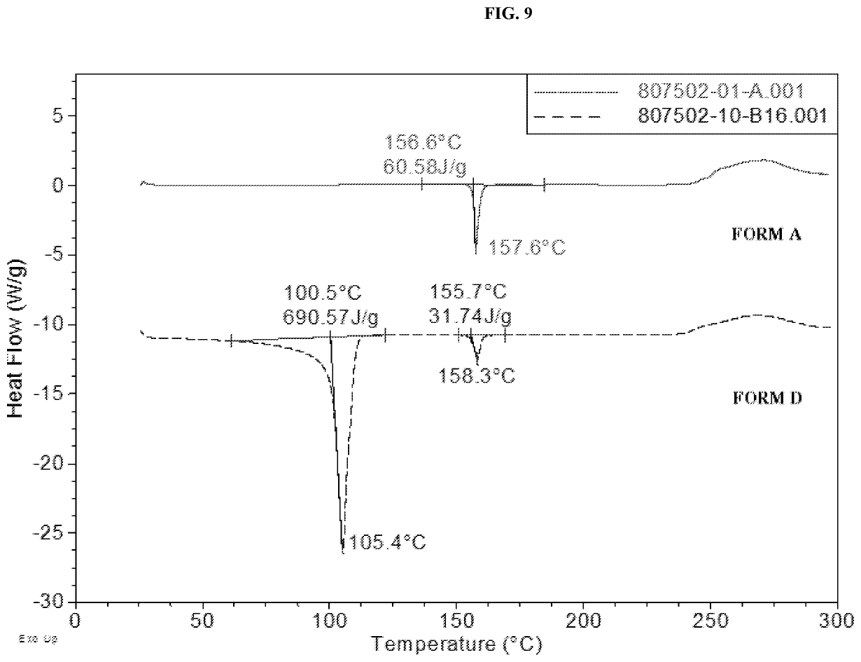

Described herein are pharmaceutical compositions comprising crystalline N-(5-(6-chloro-2,2-difluorobenzo[d][1,3]dioxol-5-yl)pyrazin-2-yl)-2-fluoro-6-methylbenzamide free base Form D which has at least one of the following properties:
(a) an X-Ray powder diffraction (XRPD) pattern substantially the same as shown in FIG. 8;
(b) an X-ray powder diffraction (XRPD) pattern comprising characteristic peaks at about 13.9° 2-Theta, about 14.4° 2-Theta, about 19.0° 2-Theta, about 19.2° 2-Theta, about 19.6° 2-Theta, about 20.0° 2-Theta, about 22.8° 2-Theta, about 25.3° 2-Theta, about 26.4° 2-Theta, and about 30.4° 2-Theta;
(c) a DSC thermogram substantially similar to the one set forth in FIG. 9; or
(d) a DSC thermogram with an endotherm having a peak at about 100.5° C. and about 155.7° C.

Described herein are pharmaceutical compositions comprising crystalline N-(5-(6-chloro-2,2-difluorobenzo[d][1,3]dioxol-5-yl)pyrazin-2-yl)-2-fluoro-6-methylbenzamide free base Form A substantially free of crystalline Form B, crystalline Form C, crystalline Form D, or any combination thereof. In some embodiments, the pharmaceutical compositions comprising crystalline N-(5-(6-chloro-2,2-difluorobenzo[d][1,3]dioxol-5-yl)pyrazin-2-yl)-2-fluoro-6-methylbenzamide free base Form A is substantially free of crystalline Form B. In some embodiments, the pharmaceutical compositions comprising crystalline N-(5-(6-chloro-2,2-difluorobenzo[d][1,3]dioxol-5-yl)pyrazin-2-yl)-2-fluoro-6-methylbenzamide free base Form A is substantially free of crystalline Form C. In some embodiments, the pharmaceutical compositions comprising crystalline N-(5-(6-chloro-2,2-difluorobenzo[d][1,3]dioxol-5-yl)pyrazin-2-yl)-2-fluoro-6-methylbenzamide free base Form A is substantially free of crystalline Form D.

### Emulsion

Described herein is a pharmaceutical composition in the form of an emulsion. In some embodiments, the emulsion comprises two immiscible phases: an aqueous phase and an oil phase. In some embodiments, the emulsion comprises N-(5-(6-chloro-2,2-difluorobenzo[d][1,3]dioxol-5-yl)pyrazin-2-yl)-2-fluoro-6-methylbenzamide, or a pharmaceutically acceptable salt thereof, and a pharmaceutically acceptable excipient. In some embodiments, N-(5-(6-chloro-2,2-difluorobenzo[d][1,3]dioxol-5-yl)pyrazin-2-yl)-2-fluoro-6-methylbenzamide is in the form of a free base. In some embodiments, N-(5-(6-chloro-2,2-difluorobenzo[d][1,3]dioxol-5-yl)pyrazin-2-yl)-2-fluoro-6-methylbenzamide is crystalline. In some embodiments, N-(5-(6-chloro-2,2-difluorobenzo[d][1,3]dioxol-5-yl)pyrazin-2-yl)-2-fluoro-6-methylbenzamide free base is crystalline Form A. In some embodiments, the emulsion is essentially free of crystalline form B. In some embodiments, the emulsion is suitable for injection. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is fully dissolved in the emulsion. In some embodiments, the pharmaceutically acceptable excipient is selected from an emulsifier, an oil, a tonicity adjustor, a chelating agent, a pH adjustor, and any combination thereof. In some embodiments, the pharmaceutically acceptable excipient is selected from lecithin, soybean oil (SBO), Medium Chain Triglycerides (MCT), cholesterol, Vitamin E succinate (VES), sucrose, glycerin, EDTA-Na₂, and any combination thereof. In some embodiments, the emulsion comprises lecithin, soybean oil (SBO), Medium Chain Triglycerides (MCT), cholesterol, Vitamin E succinate (VES), sucrose, glycerin, EDTA-Na₂, or any combination thereof. In some embodiments, the lecithin is egg lecithin. In some embodiments, the lecithin is soy lecithin. In some embodiments, the emulsion further comprises a pH adjustor selected from NaOH, HCl, and any combination thereof. In some embodiments, the emulsion further comprises water.

### CRAC Channel Inhibitor

In one aspect, the emulsion described herein comprises N-(5-(6-chloro-2,2-difluorobenzo[d][1,3]dioxol-5-yl)pyrazin-2-yl)-2-fluoro-6-methylbenzamide, or a pharmaceutically acceptable salt thereof. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1 mg/mL to about 4.0 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, or about 4 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1 mg/mL to about 3.0 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1 mg/mL to about 2.0 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 1.0 mg/mL to about 2.0 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 1.0 mg/mL to about 1.8 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 1.0 mg/mL to about 1.6 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of less than about 1.8 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of about 1.6 mg/mL in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of less than about 1.8 mg/mL in the emulsion to avoid precipitation of crystalline Form B. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1% to about 1% (w/w) in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of about 0.1%, about 0.2%, about 0.3%, about 0.4%, about 0.5%, about 0.6%, about 0.7%, about 0.8%, about 0.9%, or about 1% (w/w) in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1% to about 0.3% (w/w) in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1% to about 0.25% (w/w) in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1% to about 0.18% (w/w) in the emulsion. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 0.1% to about 0.16% (w/w) in the emulsion.

### Oil

In one aspect, the emulsion described herein comprises an oil. The oil in the emulsion is any pharmaceutical-grade oil, preferably triglycerides such as, but not limited to soybean oil (SBO), safflower seed oil, olive oil, cottonseed oil, sunflower oil, fish oil (containing the omega-3 fatty acids eicosapentaenoic acid (EPA), and docosahexaenoic acid (DHA)), castor oil, sesame oil, peanut oil, corn oil, medium chain triglycerides (MCT), and any combination thereof. In some embodiments, the oil is medium chain triglycerides (MCT). In some embodiments, the oil is soybean oil (SBO). In some embodiments, the oil is present at a concentration from about 1% to about 10% (w/w) in the emulsion. In some embodiments, the oil is present at a concentration of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% (w/w) in the emulsion. In some embodiments, the oil is present at a concentration from about 1% to about 5% (w/w) in the emulsion. In some embodiments, the oil is present at a concentration from about 5% to about 10% (w/w) in the emulsion. In some embodiments, the oil is present at a concentration from about 3% to about 7% (w/w) in the emulsion. In some embodiments, the oil is present at a concentration of about 5% (w/w) in the emulsion. In some embodiments, the oil is medium chain triglycerides (MCT) and is present at a concentration of about 5% (w/w) in the emulsion.

### Emulsifier

In one aspect, the emulsion described herein comprises an emulsifier. In some embodiments, the process of coalescence is reduced by the addition of an emulsifier in addition to the oil and the aqueous solvent. In some embodiments, the emulsifier is surface active and reduces surface tension to below about 10 dynes/cm. In some embodiments, the emulsifier is absorbed quickly around the dispersed drops as a condensed, non-adherent film to prevent coalescence. In some embodiments, the emulsifier imparts to the droplet an adequate electrical potential so that mutual repulsion occurs. In some embodiments, the emulsifier increases the viscosity of the emulsion. Exemplary emulsifiers are, without limitation: potassium laurate, triethanolamine stearate, sodium lauryl sulfate, alkyl polyoxyethylene sulfates, dioctyl sodium sulfosuccinate, cetyltrimethylammonium bromide, lauryldimethylbenzyl ammonium chloride, sorbitan fatty acid esters, polyoxyethylene, polyoxyethylene fatty alcohol ethers, polyoxyethylene sorbitan fatty acid esters, polyoxyethylene/polyoxypropylene block copolymer (poloxamer), lanolin alcohols, acacia, gelatin, lecithin, cholesterol, and any combination thereof. In some embodiments, the emulsifier is lecithin. Lecithin is a generic term to designate any group of yellow-brownish fatty substances occurring in animal and plant tissues, which are amphiphilic; they attract both water and fatty substances (and so are both hydrophilic and lipophilic). Lecithins are usually phospholipids, composed of phosphoric acid with choline, glycerol, or other fatty acids usually glycolipids or triglyceride. Glycerophospholipids in lecithin include phosphatidylcholine, phosphatidylethanolamine, phosphatidylinositol, phosphatidylserine, and phosphatidic acid. In some embodiments, the lecithin is egg lecithin. In some embodiments, the lecithin is soy lecithin. In some embodiments, the emulsifier is present at a concentration from about 5% to about 15% (w/w) in the emulsion. In some embodiments, the emulsifier is present at a concentration of about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% (w/w) in the emulsion. In some embodiments, the emulsifier is present at a concentration from about 5% to about 10% (w/w) in the emulsion. In some embodiments, the emulsifier is present at a concentration from about 10% to about 15% (w/w) in the emulsion. In some embodiments, the emulsifier is present at a concentration from about 8% to about 12% (w/w) in the emulsion. In some embodiments, the emulsifier is present at a concentration of about 10% (w/w) in the emulsion. In some embodiments, the emulsifier is lecithin and is present at a concentration of about 10% (w/w) in the emulsion.

### Tonicity Adjustor

In one aspect, the emulsion described herein comprises a tonicity adjustor. In some embodiments, the emulsion described herein is isotonic. Tonicity adjustors include, but are not limited to, dextrose, glycerin, sucrose, mannitol, potassium chloride, sodium chloride, and any combination thereof. In some embodiments, the tonicity adjustor is glycerin. In some embodiments, the tonicity adjustor is sucrose. In some embodiments, the tonicity adjustor is present at a concentration from about 1% to about 5% (w/w) in the emulsion. In some embodiments, the tonicity adjustor is present at a concentration of about 1%, about 1.5%, about 2%, about 2.5%, about 3%, about 3.5%, about 4%, about 4.5%, or about 5% (w/w) in the emulsion. In some embodiments, the tonicity adjustor is present at a concentration from about 1% to about 2.5% (w/w) in the emulsion. In some embodiments, the tonicity adjustor is present at a concentration from about 2.5% to about 5% (w/w) in the emulsion. In some embodiments, the tonicity adjustor is present at a concentration from about 2% to about 4% (w/w) in the emulsion. In some embodiments, the tonicity adjustor is present at a concentration of about 2.25% (w/w) in the emulsion. In some embodiments, the tonicity adjustor is glycerin and is present at a concentration of about 2.25% (w/w) in the emulsion.

### Chelating Agent

In one aspect, the emulsion described herein comprises a chelating agent. In some embodiments, the chelating agent is EDTA. In some embodiments, the chelating agent is EDTA-Na₂. In some embodiments, the tonicity adjustor is present at a concentration from about 0.001% to about 0.01% (w/w) in the emulsion. In some embodiments, the chelating agent is present at a concentration of about 0.001%, about 0.002%, about 0.003%, about 0.004%, about 0.005%, about 0.006%, about 0.007%, about 0.008%, about 0.009%, or about 0.01% (w/w) in the emulsion. In some embodiments, the chelating agent is present at a concentration from about 0.001% to about 0.005% (w/w) in the emulsion. In some embodiments, the chelating agent is present at a concentration from about 0.005% to about 0.01% (w/w) in the emulsion. In some embodiments, the chelating agent is present at a concentration of about 0.005% (w/w) in the emulsion. In some embodiments, the chelating agent is present at a concentration of about 0.0055% (w/w) in the emulsion. In some embodiments, the chelating agent is EDTA-Na₂ and is present at a concentration of about 0.0055% (w/w) in the emulsion.

### Additional Excipients

In some embodiments, the emulsion further contains co-solvents or other solubility enhancers, preservatives (exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (such as methylparaben, ethylparaben, propylparaben, butylparaben, and their salts), benzoic acid, sodium benzoate, potassium sorbate, vanillin, and the like), antioxidants, stabilizers, pH-adjusting agents (NaOH or HCl), polymers as suspending agents, sweeteners, and any combination thereof. These additional excipients are selected based on function and compatibility with the pharmaceutical composition described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999), herein incorporated by reference as they relate to excipients and emulsion formulation.

### pH of the Emulsion

In one aspect, the pH of the emulsions described herein is adjusted with one or more pH adjustors. Non-limiting examples of pH adjustors include, but are not limited to, sodium hydroxide (NaOH) and hydrochloric acid (HCl). In some embodiments, the pH of the emulsion described herein is from about 4 to about 9. In some embodiments, the pH of the emulsion described herein is about 4, about 4.5, about 5, about 5.5, about 6, about 6.5, about 7, about 7.5, about 8, about 8.5, or about 9. In some embodiments, the pH of the emulsion described herein is from about 6 to about 8. In some embodiments, the pH of the emulsion described herein is from about 6 to about 7. In some embodiments, the pH of the emulsion described herein is from about 7 to about 8. In some embodiments, the pH of the emulsion described herein is about 7.

### Mean Droplet Size

In one aspect, the emulsion is a mixture of two immiscible liquids (an organic “oil” and water) in which one liquid (the dispersed phase) is in the form of microscopic droplets dispersed in the other (continuous) phase. In some embodiments, the mean droplet size is from about 100 to about 500 nm. In some embodiments, the mean droplet size is about 100 nm, about 150 nm, about 200 nm, about 250 nm, about 300 nm, about 350 nm, about 400 nm, about 450 nm, or about 500 nm. In some embodiments, the mean droplet size is less than 200 nm.

### Stability of the Emulsion

### Chemical Stability:

The N-(5-(6-chloro-2,2-difluorobenzo[d][1,3]dioxol-5-yl)pyrazin-2-yl)-2-fluoro-6-methylbenzamide emulsions described herein are stable in various storage conditions including refrigerated, ambient, and accelerated conditions. In some embodiments, a stable N-(5-(6-chloro-2,2-difluorobenzo[d][1,3]dioxol-5-yl)pyrazin-2-yl)-2-fluoro-6-methylbenzamide emulsion as used herein refers to an emulsion having about 80% or greater of the initial N-(5-(6-chloro-2,2-difluorobenzo[d][1,3]dioxol-5-yl)pyrazin-2-yl)-2-fluoro-6-methylbenzamide amount. In some embodiments, a stable N-(5-(6-chloro-2,2-difluorobenzo[d][1,3]dioxol-5-yl)pyrazin-2-yl)-2-fluoro-6-methylbenzamide emulsion as used herein refers to an emulsion having about 4% (w/w) or less total related substances at the end of a given storage period. The percentage of related substances is calculated from the amount of related substances relative to the amount of N-(5-(6-chloro-2,2-difluorobenzo[d][1,3]dioxol-5-yl)pyrazin-2-yl)-2-fluoro-6-methylbenzamide. Stability is assessed by HPLC or any other known testing method. In some embodiments, the stable N-(5-(6-chloro-2,2-difluorobenzo[d][1,3]dioxol-5-yl)pyrazin-2-yl)-2-fluoro-6-methylbenzamide emulsion comprises about 4% (w/w), about 3% (w/w), about 2.5% (w/w), about 2% (w/w), about 1.5% (w/w), about 1% (w/w), about 0.9% (w/w), about 0.8% (w/w), about 0.7% (w/w), about 0.6% (w/w), about 0.5% (w/w), about 0.4% (w/w), about 0.3% (w/w), about 0.2% (w/w), or about 0.1% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide emulsion comprises about 4% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide emulsion comprises about 3% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide emulsion comprises about 2% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide emulsion comprises about 1% (w/w) total related substances. At refrigerated (5±3° C.) and ambient conditions, the N-(5-(6-chloro-2,2-difluorobenzo[d][1,3]dioxol-5-yl)pyrazin-2-yl)-2-fluoro-6-methylbenzamide emulsions described herein are stable for at least 1 month, at least 2 months, at least 3 months, at least 6 months, at least 9 months, at least 12 months, at least 15 months, at least 18 months, at least 24 months, at least 30 months, or at least 36 months. At accelerated conditions, the N-(5-(6-chloro-2,2-difluorobenzo[d][1,3]dioxol-5-yl)pyrazin-2-yl)-2-fluoro-6-methylbenzamide emulsions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

### Physical Stability:

The physical stability of the emulsion is associated with three major phenomena:

### (1) Creaming or Sedimentation:

Creaming is the upward movement of dispersed droplets relative to the continuous phase. Sedimentation, the reverse process, is the downward movement of particles. In any emulsion, one process or the other takes place depending on the densities of the dispersed and continuous phases. In some embodiments, the emulsion described herein does not show any creaming for at least 1 month, at least 2 months, at least 3 months, at least 6 months, at least 9 months, at least 12 months, at least 15 months, at least 18 months, at least 24 months, at least 30 months, or at least 36 months. In some embodiments, the emulsion described herein does not show any sedimentation for at least 1 month, at least 2 months, at least 3 months, at least 6 months, at least 9 months, at least 12 months, at least 15 months, at least 18 months, at least 24 months, at least 30 months, or at least 36 months.

### (2) Aggregation and Coalescence:

Aggregation (or flocculation) is a process wherein the dispersed droplets come together but do not fuse. Coalescence is a process wherein the droplets completely fuse which leads to a decrease in the number of droplets and the ultimate separation of the two immiscible phases. Aggregation precedes coalescence but coalescence does not necessarily follow from aggregation. In some embodiments, the emulsion described herein does not show any aggregation for at least 1 month, at least 2 months, at least 3 months, at least 6 months, at least 9 months, at least 12 months, at least 15 months, at least 18 months, at least 24 months, at least 30 months, or at least 36 months. In some embodiments, the emulsion described herein does not show any coalescence for at least 1 month, at least 2 months, at least 3 months, at least 6 months, at least 9 months, at least 12 months, at least 15 months, at least 18 months, at least 24 months, at least 30 months, or at least 36 months.

### (3) Inversion:

An emulsion is said to invert when it changes from an O/W (oil in water) emulsion to become a W/O (water in oil) emulsion and vice versa. In some embodiments, the emulsion described herein does not show any sign of inversion for at least 1 month, at least 2 months, at least 3 months, at least 6 months, at least 9 months, at least 12 months, at least 15 months, at least 18 months, at least 24 months, at least 30 months, or at least 36 months.

### Powder for Reconstitution/Nanosuspension

Described herein is a pharmaceutical composition in the form of a powder for reconstitution. In some embodiments, the powder for reconstitution is reconstituted with an aqueous carrier to form a nanosuspension. In some embodiments, the nanosuspension comprises nanoparticles. In some embodiments, the aqueous carrier is selected from water, saline, 5% dextrose in water, 5% dextrose in saline, and any combination thereof. In some embodiments, the aqueous carrier is water. In some embodiments, the powder for reconstitution comprises N-(5-(6-chloro-2,2-difluorobenzo[d][1,3]dioxol-5-yl)pyrazin-2-yl)-2-fluoro-6-methylbenzamide, or a pharmaceutically acceptable salt thereof, and a pharmaceutically acceptable excipient. In some embodiments, N-(5-(6-chloro-2,2-difluorobenzo[d][1,3]dioxol-5-yl)pyrazin-2-yl)-2-fluoro-6-methylbenzamide is in the form of a free base. In some embodiments, N-(5-(6-chloro-2,2-difluorobenzo[d][1,3]dioxol-5-yl)pyrazin-2-yl)-2-fluoro-6-methylbenzamide is crystalline. In some embodiments, N-(5-(6-chloro-2,2-difluorobenzo[d][1,3]dioxol-5-yl)pyrazin-2-yl)-2-fluoro-6-methylbenzamide free base is crystalline Form A. In some embodiments, the nanosuspension is essentially free of crystalline form B. In some embodiments, the nanosuspension is suitable for injection. In some embodiments, the pharmaceutically acceptable excipient is a stabilizing agent. In some embodiments, the stabilizing agent is a surfactant or a polymer surfactant. In some embodiments, the pharmaceutically acceptable excipient is selected from polyvinylpyrrolidone (PVP), sodium deoxycholate, and any combination thereof. In some embodiments, the powder for reconstitution further comprises a cryoprotectant. In some embodiments, the cryoprotectant is selected from sucrose, sucrose/mannitol, trehalose, trehalose/mannitol, and any combination thereof. In some embodiments, the cryoprotectant system is sucrose.

### CRAC Channel Inhibitor

In one aspect, the powder for reconstitution described herein comprises N-(5-(6-chloro-2,2-difluorobenzo[d][1,3]dioxol-5-yl)pyrazin-2-yl)-2-fluoro-6-methylbenzamide, or a pharmaceutically acceptable salt thereof. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 1 mg/mL to about 100 mg/mL in the nanosuspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL in the nanosuspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 1 mg/mL to about 10 mg/mL in the nanosuspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 50 mg/mL to about 100 mg/mL in the nanouspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 30 mg/mL to about 70 mg/mL in the nanosuspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration from about 40 mg/mL to about 60 mg/mL in the nanosuspension once reconstituted. In some embodiments, N-(5-(6-chloro-2,2-difluorobenzo[d][1,3]dioxol-5-yl)pyrazin-2-yl)-2-fluoro-6-methylbenzamide, or a pharmaceutically acceptable salt thereof, is present at a concentration of about 50 mg/mL in the nanosuspension, once reconstituted.

### Stabilizing Agents

The nanosuspensions described herein comprise a stabilizing agent to stabilize the nanosuspension by preventing agglomeration of the nanoparticles in the solution and by preventing or minimizing the formation of large particles, i.e., particles with dimensions >1 m. Examples of such stabilizing agents are well known to a person of skill in the art. In some embodiments, the stabilizing agent is a surfactant, surfactant polymer, or any combination thereof. In some embodiments, the stabilizing agent is water soluble. Suitable surfactants for use in the nanosuspension of the invention include, but are not limited to, polysorbate surfactants, poloxamer surfactants, dioctyl sodium sulfosuccinate (DOSS), sodium deoxycholate, or any combination thereof. A typical polysorbate surfactant is Tween (Registered trademark), for example Tween 20 (Registered trademark) or Tween 80 (Registered trademark). Typical poloxamer surfactants include poloxamer 188 and poloxamer 228. Polyvinylpyrrolidone (also known as Povidone or PVP) is a water soluble polymer made from the monomer of N-vinylpyrrolidone. A suitable surfactant polymer is polyvinylpyrrolidone (PVP). PVP is often defined in terms of a K-value which characterises the mean molecular weight e.g. Povidone K 12, Povidone K 17, Povidone K 25, Povidone K 30 and Povidone K 90. PVP is available under various trade names including Plasdone C-15 (Registered trademark), Kollidon 12PF (Registered trademark), Kollidon 17PF (Registered trademark) and Kollidon 30 (Registered trademark). In one embodiment, the PVP has a mean molecular weight of between about 2,000 Da and 1,500,000 Da, such as between about 2,000 Da and about 5,000 Da; between about 6,000 Da and about 12,000 Da; between about 25,000 Da and about 40,000 Da; between about 41,000 Da and about 65,000 Da or between about 1,000,000 Da and about 1,500,000 Da. Suitably, the PVP has a mean molecular weight between about 2,000 Da and about 3000 Da (corresponding to Kollidon 12).

In one aspect, the powder for reconstitution described herein comprises a stabilizing agent. In some embodiments, the stabilizing agent is polyvinylpyrrolidone (PVP) and is present at a concentration from about 0.1% to about 5% (w/w) in the powder for reconstitution. In some embodiments, polyvinylpyrrolidone (PVP) is present at a concentration of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% (w/w) in the powder for reconstitution. In some embodiments, polyvinylpyrrolidone (PVP) is present at a concentration from about 0.1% to about 2.5% (w/w) in the powder for reconstitution. In some embodiments, polyvinylpyrrolidone (PVP) is present at a concentration from about 0.1% to about 0.5% (w/w) in the powder for reconstitution. In some embodiments, polyvinylpyrrolidone (PVP) is present at a concentration of about 0.5% (w/w) in the powder for reconstitution.

In one aspect, the powder for reconstitution described herein comprises a second stabilizing agent. In some embodiments, the second stabilizing agent is sodium deoxycholate and is present at a concentration from about 0.1% to about 5% (w/w) in the powder for reconstitution. In some embodiments, sodium deoxycholate is present at a concentration of about 0.1%, about 0.2%, about 0.2%, about 0.3%, about 0.4%, about 0.5%, about 0.6%, about 0.7%, about 0.8%, about 0.9%, or about 1% (w/w) in the powder for reconstitution. In some embodiments, sodium deoxycholate is present at a concentration from about 0.1% to about 0.5% (w/w) in the powder for reconstitution. In some embodiments, sodium deoxycholate is present at a concentration from about 0.1% to about 0.2% (w/w) in the powder for reconstitution. In some embodiments, sodium deoxycholate is present at a concentration of about 0.125% (w/w) in the powder for reconstitution.

### Cryoprotectant

In one aspect, the powder for reconstitution described herein comprises a cryoprotectant. In some embodiments, the powder for reconstitution comprises nanoparticles. In some embodiments, the nanoparticles are prepared in a liquid medium and a drying method such as freeze-drying. When the dried form is reconstituted in an aqueous carrier, it is redispersed to achieve its original particle size. In some embodiments, the redispersibility of the dried nanoparticles depends on the parameters of the freeze-drying process. In some embodiments, the redispersibility of the dried nanoparticles depends on the use of a cryoprotectant. Exemplary cryoprotectants are, without limitation: sucrose, lactose, mannitol, trehalose, sucrose/mannitol, trehalose/mannitol, polyethylene glycol, and any combination thereof. In some embodiments, the cryoprotectant is sucrose. In some embodiments, the cryoprotectant is present at a concentration from about 1% to about 20% (w/w) in the powder for reconstitution. In some embodiments, the cryoprotectant is present at a concentration of about about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% (w/w) in the powder for reconstitution. In some embodiments, the cryoprotectant is present at a concentration from about 1% to about 10% (w/w) in the powder for reconstitution. In some embodiments, the cryoprotectant is present at a concentration from about 10% to about 20% (w/w) in the powder for reconstitution. In some embodiments, the cryoprotectant is present at a concentration from about 8% to about 12% (w/w) in the powder for reconstitution. In some embodiments, the cryoprotectant is present at a concentration of about 10% (w/w) in the powder for reconstitution.

### Additional Excipients

In some embodiments, the powder for reconstitution further contains preservatives (exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (such as methylparaben, ethylparaben, propylparaben, butylparaben and their salts), benzoic acid, sodium benzoate, potassium sorbate, vanillin, and the like), antioxidants, glidants, disintegrants, stabilizers, sweeteners, and any combination thereof. These additional excipients are selected based on function and compatibility with the pharmaceutical composition described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999), herein incorporated by reference as they relate to excipients and powder for reconstitution or nanosuspension formulation.

### pH of the Nanosuspension

In one aspect, the powder for reconstitution is reconstituted with an aqueous carrier. In some embodiments, the pH of the nanosuspension described herein is from about 4 to about 9. In some embodiments, the pH of the nanosuspension described herein is about 4, about 4.5, about 5, about 5.5, about 6, about 6.5, about 7, about 7.5, about 8, about 8.5, or about 9. In some embodiments, the pH of the nanosuspension described herein is from about 6 to about 8. In some embodiments, the pH of the nanosuspension described herein is from about 6 to about 7. In some embodiments, the pH of the nanosuspension described herein is from about 7 to about 8. In some embodiments, the pH of the nanosuspension described herein is about 7.

### Nanoparticle Size

In one aspect, the powder for reconstitution and nanosuspension comprise nanoparticles. In some embodiments, the average nanoparticle diameter is from about 50 nm to about 500 nm. In some embodiments, the mean droplet size is about 100 nm, about 150 nm, about 200 nm, about 250 nm, about 300 nm, about 350 nm, about 400 nm, about 450 nm, or about 500 nm. In some embodiments, the mean droplet size is less than 200 nm.

### Stability of the Powder for Reconstitution

The N-(5-(6-chloro-2,2-difluorobenzo[d][1,3]dioxol-5-yl)pyrazin-2-yl)-2-fluoro-6-methylbenzamide powders for reconstitution described herein are stable in various storage conditions including refrigerated, ambient, and accelerated conditions. In some embodiments, a stable N-(5-(6-chloro-2,2-difluorobenzo[d][1,3]dioxol-5-yl)pyrazin-2-yl)-2-fluoro-6-methylbenzamide powder for reconstitution as used herein refers to a powder for reconstitution having about 80% or greater of the initial N-(5-(6-chloro-2,2-difluorobenzo[d][1,3]dioxol-5-yl)pyrazin-2-yl)-2-fluoro-6-methylbenzamide amount. In some embodiments, a stable N-(5-(6-chloro-2,2-difluorobenzo[d][1,3]dioxol-5-yl)pyrazin-2-yl)-2-fluoro-6-methylbenzamide powder for reconstitution as used herein refers to a powder for reconstitution having about 4% (w/w) or less total related substances at the end of a given storage period. The percentage of related substances is calculated from the amount of related substances relative to the amount of N-(5-(6-chloro-2,2-difluorobenzo[d][1,3]dioxol-5-yl)pyrazin-2-yl)-2-fluoro-6-methylbenzamide.

Stability is assessed by HPLC or any other known testing method. In some embodiments, the stable N-(5-(6-chloro-2,2-difluorobenzo[d][1,3]dioxol-5-yl)pyrazin-2-yl)-2-fluoro-6-methylbenzamide powder for reconstitution comprises about 4% (w/w), about 3% (w/w), about 2.5% (w/w), about 2% (w/w), about 1.5% (w/w), about 1% (w/w), about 0.9% (w/w), about 0.8% (w/w), about 0.7% (w/w), about 0.6% (w/w), about 0.5% (w/w), about 0.4% (w/w), about 0.3% (w/w), about 0.2% (w/w), or about 0.1% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide powder for reconstitution comprises about 4% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide powder for reconstitution comprises about 3% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide powder for reconstitution comprises about 2% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide powder for reconstitution comprises about 1% (w/w) total related substances. At refrigerated (5±3° C.) and ambient conditions, the N-(5-(6-chloro-2,2-difluorobenzo[d][1,3]dioxol-5-yl)pyrazin-2-yl)-2-fluoro-6-methylbenzamide powders for reconstitution described herein are stable for at least 1 month, at least 2 months, at least 3 months, at least 6 months, at least 9 months, at least 12 months, at least 15 months, at least 18 months, at least 24 months, at least 30 months, or at least 36 months. At accelerated conditions, the N-(5-(6-chloro-2,2-difluorobenzo[d][1,3]dioxol-5-yl)pyrazin-2-yl)-2-fluoro-6-methylbenzamide powders for reconstitution described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

### Stability of the Nanosuspension

The N-(5-(6-chloro-2,2-difluorobenzo[d][1,3]dioxol-5-yl)pyrazin-2-yl)-2-fluoro-6-methylbenzamide nanosuspensions described herein are stable in various storage conditions including refrigerated, ambient, and accelerated conditions. In some embodiments, a stable N-(5-(6-chloro-2,2-difluorobenzo[d][1,3]dioxol-5-yl)pyrazin-2-yl)-2-fluoro-6-methylbenzamide nanosuspension as used herein refers to a nanosuspension having about 80% or greater of the initial N-(5-(6-chloro-2,2-difluorobenzo[d][1,3]dioxol-5-yl)pyrazin-2-yl)-2-fluoro-6-methylbenzamide amount. In some embodiments, a stable N-(5-(6-chloro-2,2-difluorobenzo[d][1,3]dioxol-5-yl)pyrazin-2-yl)-2-fluoro-6-methylbenzamide nanosuspension as used herein refers to a nanosuspension having about 4% (w/w) or less total related substances at the end of a given storage period. The percentage of related substances is calculated from the amount of related substances relative to the amount of N-(5-(6-chloro-2,2-difluorobenzo[d][1,3]dioxol-5-yl)pyrazin-2-yl)-2-fluoro-6-methylbenzamide. Stability is assessed by HPLC or any other known testing method. In some embodiments, the stable N-(5-(6-chloro-2,2-difluorobenzo[d][1,3]dioxol-5-yl)pyrazin-2-yl)-2-fluoro-6-methylbenzamide nanosuspension comprises about 4% (w/w), about 3% (w/w), about 2.5% (w/w), about 2% (w/w), about 1.5% (w/w), about 1% (w/w), about 0.9% (w/w), about 0.8% (w/w), about 0.7% (w/w), about 0.6% (w/w), about 0.5% (w/w), about 0.4% (w/w), about 0.3% (w/w), about 0.2% (w/w), or about 0.1% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide nanosuspension comprises about 4% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide nanosuspension comprises about 3% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide nanosuspension comprises about 2% (w/w) total related substances. In yet other embodiments, the stable N-(5-(6-chloro-2,2-difluorobenzo[d][1,3]dioxol-5-yl)pyrazin-2-yl)-2-fluoro-6-methylbenzamide nanosuspension comprises about 1% (w/w) total related substances. At refrigerated (5±3° C.) and ambient conditions, the N-(5-(6-chloro-2,2-difluorobenzo[d][1,3]dioxol-5-yl)pyrazin-2-yl)-2-fluoro-6-methylbenzamide nanosuspensions described herein are stable for at least 1 month, at least 2 months, at least 3 months, at least 6 months, at least 9 months, at least 12 months, at least 15 months, at least 18 months, at least 24 months, at least 30 months, or at least 36 months. At accelerated conditions, the N-(5-(6-chloro-2,2-difluorobenzo[d][1,3]dioxol-5-yl)pyrazin-2-yl)-2-fluoro-6-methylbenzamide nanosuspensions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

### Methods

Provided herein, are methods of treatment comprising administration of the pharmaceutical compositions described herein to a subject.

Described herein are pharmaceutical compositions for modulating intracellular calcium to ameliorate or prevent symptoms of pancreatitis. In some aspects, the pancreatitis is acute pancreatitis. In some aspects, the pancreatitis is chronic pancreatitis.

Described herein are pharmaceutical compositions for modulating intracellular calcium to ameliorate or prevent symptoms of a viral disease. In some aspects, the viral disease is a hemorrhagic fever virus. In some aspects, the hemorrhagic fever virus is an arenavirus, a filovirus, a bunyavirus, a flavivirus, a rhabdovirus, or combinations thereof. Hemorrhagic fever viruses include, by way of non-limiting examples, Ebola virus, Marburg virus, Lassa virus, Junin virus, Rotavirus, West Nile virus, Zika virus, Coxsackievirus, Hepatitis B virus, Epstein Barr virus.

Described herein are pharmaceutical compositions for modulating intracellular calcium to ameliorate or prevent symptoms of Th17-induced diseases. In some aspects, the Th17-induced disease is an inflammatory disease. In further aspects, the Th17-induced disease is an autoimmune disorder.

Described herein are pharmaceutical compositions for modulating intracellular calcium to ameliorate or prevent fibrosis. In some embodiments, the fibrosis is a pulmonary fibrosis. In some embodiments, the pulmonary fibrosis is idiopathic pulmonary fibrosis (IPF). In some embodiments, the pulmonary fibrosis is cystic fibrosis. In some embodiments, the fibrosis is a liver fibrosis. In some embodiments, the liver fibrosis is cirrhosis. In some embodiments, the fibrosis is atrial fibrosis, endomyocardial fibrosis, old myocardial infarction, glial scar, arthrofibrosis, crohn's disease, Dupuytren's contracture, keloid, mediastinal fibrosis, myelofibrosis, peyronie's disease, nephrogenic systemic fibrosis, progressive massive fibrosis, retroperitoneal fibrosis, or scleroderma/systemic sclerosis.

Described herein are pharmaceutical compositions for modulating intracellular calcium to ameliorate or prevent non-alcoholic fatty liver disease (NAFLD). In some embodiments, the non-alcoholic fatty liver disease (NAFLD) is non-alcoholic steatohepatitis (NASH).

Described herein are pharmaceutical compositions for modulating intracellular calcium to ameliorate or prevent stroke

Described herein are pharmaceutical compositions for modulating intracellular calcium to ameliorate or prevent traumatic brain injury.

### Dosage Parameters

In one aspect, the pharmaceutical compositions described herein are used for the treatment of diseases and conditions described herein. In addition, methods for treating any of the diseases or conditions described herein in a subject in need of such treatment involve administration of the pharmaceutical compositions described herein in therapeutically effective amounts to said subject.

Dosages of the pharmaceutical compositions described herein are determined by any suitable method. In some embodiments, maximum tolerated doses (MTD) and maximum response doses (MRD) for N-(5-(6-chloro-2,2-difluorobenzo[d][1,3]dioxol-5-yl)pyrazin-2-yl)-2-fluoro-6-methylbenzamide are determined via established animal and human experimental protocols. In some embodiments, toxicity and therapeutic efficacy of N-(5-(6-chloro-2,2-difluorobenzo[d][1,3]dioxol-5-yl)pyrazin-2-yl)-2-fluoro-6-methylbenzamide is determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in humans. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols. In other embodiments, the pharmaceutical compositions are provided at the maximum tolerated dose (MTD) for N-(5-(6-chloro-2,2-difluorobenzo[d][1,3]dioxol-5-yl)pyrazin-2-yl)-2-fluoro-6-methylbenzamide. In other embodiments, the amount of the pharmaceutical composition administered is from about 10% to about 90% of the maximum tolerated dose (MTD), from about 25% to about 75% of the MTD, or about 50% of the MTD for N-(5-(6-chloro-2,2-difluorobenzo[d][1,3]dioxol-5-yl)pyrazin-2-yl)-2-fluoro-6-methylbenzamide. In particular embodiments, the amount of the pharmaceutical compositions administered is about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD for N-(5-(6-chloro-2,2-difluorobenzo[d][1,3]dioxol-5-yl)pyrazin-2-yl)-2-fluoro-6-methylbenzamide. In some embodiments, the pharmaceutical compositions are provided at a dose ranging from about 0.5 mg/kg to about 25 mg/kg. In some embodiments, the pharmaceutical compositions are provided at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 10.5 mg/kg, about 11 mg/kg, about 11.5 mg/kg, about 12 mg/kg, about 12.5 mg/kg, about 13 mg/kg, about 13.5 mg/kg, about 14 mg/kg, about 14.5 mg/kg, about 15 mg/kg, about 15.5 mg/kg, about 16 mg/kg, about 16.5 mg/kg, about 17 mg/kg, about 17.5 mg/kg, about 18 mg/kg, about 18.5 mg/kg, about 19 mg/kg, about 19.5 mg/kg, about 20 mg/kg, about 20.5 mg/kg, about 21 mg/kg, about 21.5 mg/kg, about 22 mg/kg, about 22.5 mg/kg, about 23 mg/kg, about 23.5 mg/kg, about 24 mg/kg, about 24.5 mg/kg, or about 25 mg/kg. In some embodiments, the pharmaceutical compositions are provided at a dose ranging from about 0.5 mg/kg to about 3.5 mg/kg. In some embodiments, the pharmaceutical compositions are provided at a dose ranging from about 0.5 mg/kg to about 5 mg/kg. In some embodiments, the pharmaceutical compositions are provided at a dose ranging from about 0.5 mg/kg to about 10 mg/kg.

In some embodiments, the pharmaceutical composition comprises N-(5-(6-chloro-2,2-difluorobenzo[d][1,3]dioxol-5-yl)pyrazin-2-yl)-2-fluoro-6-methylbenzamide in an amount from about 0.1 mg/mL to about 4 mg/mL. In specific embodiments, the composition comprises N-(5-(6-chloro-2,2-difluorobenzo[d][1,3]dioxol-5-yl)pyrazin-2-yl)-2-fluoro-6-methylbenzamide in an amount of less than about 1.8 mg/mL. In other embodiments, the composition comprises N-(5-(6-chloro-2,2-difluorobenzo[d][1,3]dioxol-5-yl)pyrazin-2-yl)-2-fluoro-6-methylbenzamide in an amount of about 1.6 mg/mL. In some embodiments, the pharmaceutical composition comprises N-(5-(6-chloro-2,2-difluorobenzo[d][1,3]dioxol-5-yl)pyrazin-2-yl)-2-fluoro-6-methylbenzamide in an amount from about 0.1 mg/mL to about 100 mg/mL. In specific embodiments, the composition comprises N-(5-(6-chloro-2,2-difluorobenzo[d][1,3]dioxol-5-yl)pyrazin-2-yl)-2-fluoro-6-methylbenzamide in an amount from about 40 mg/mL to 60 mg/mL. In other embodiments, the composition comprises N-(5-(6-chloro-2,2-difluorobenzo[d][1,3]dioxol-5-yl)pyrazin-2-yl)-2-fluoro-6-methylbenzamide in an amount of about 50 mg/mL.

Administration of any pharmaceutical composition described herein follows any suitable dosing schedule. In certain embodiments, the pharmaceutical composition is administered on days 1 and 8 of each 21-day cycle. In other embodiments, the pharmaceutical composition is administered on days 1, 8, and 15 of each 28-day cycle. In some embodiments, the pharmaceutical composition is administered once weekly or twice weekly. In other embodiments, the pharmaceutical composition is administered three times weekly, four times weekly, five times weekly, six times weekly, or seven times weekly. In some embodiments, the pharmaceutical composition is administered once a day, twice a day, or once every two days. In some embodiments, the pharmaceutical composition is administered once every three days, once every four days, once every five days, or once every six days. One schedule may be preferred over another in consideration of schedules with other concomitant therapy. Doses of the composition may be held or modified, e.g., due to the observation of unacceptable side effects. In various embodiments of therapies described herein, the dosing schedule is optionally repeated, e.g., in the absence of disease progression or unacceptable side effects.

### Administration

Described herein are pharmaceutical compositions formulated as injectable pharmaceutical compositions. In some embodiments, the emulsions described herein are formulated as injectable emulsions. In some embodiments, the nanosuspensions described herein are formulated as injectable nanosuspensions. In some embodiments, the injectable pharmaceutical compositions are suitable for intravenous administration. In some embodiments, the injectable pharmaceutical compositions are suitable for intramuscular administration. In certain embodiments, the pharmaceutical compositions described herein are administered for prophylactic and/or therapeutic treatments. In certain therapeutic applications, the pharmaceutical compositions are administered to a patient already suffering from a disease in an amount sufficient to cure the disease or at least partially arrest or ameliorate the symptoms. Amounts effective for this use depend on the severity of the disease; previous therapy; the patient's health status, weight, and response to the pharmaceutical compositions; and the judgment of the treating physician. Therapeutically effective amounts are optionally determined by methods including, but not limited to, a dose escalation clinical trial.

In prophylactic applications, the pharmaceutical compositions described herein are administered to a patient susceptible to or otherwise at risk of a particular disease. Such an amount is defined to be a “prophylactically effective amount or dose.” In this use, the precise amounts also depend on the patient's state of health, weight, and the like. When used in a patient, effective amounts for this use will depend on the risk or susceptibility of developing the particular disease, previous therapy, the patient's health status and response to the pharmaceutical compositions, and the judgment of the treating physician.

In certain embodiments wherein the patient's condition does not improve, upon the doctor's discretion the administration of a pharmaceutical composition described herein is administered chronically, that is, for an extended period of time, including throughout the duration of the patient's life in order to ameliorate or otherwise control or limit the symptoms of the patient's disease. In other embodiments, administration of a pharmaceutical composition described herein continues until complete or partial response of a disease.

In certain embodiments wherein a patient's status does improve, the dose of a pharmaceutical composition described herein being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is, by way of example only, from about 10% to about 100%, including by way of example only 10%, 15%, 20%, 25%, 30%, 35%, 40%, 45%, 50%, 55%, 60%, 65%, 70%, 75%, 80%, 85%, 90%, 95%, and 100%.

In some embodiments, pharmaceutical compositions described herein are administered chronically. For example, in some embodiments, a pharmaceutical composition described herein is administered as a continuous dose, i.e., administered daily to a subject. In some other embodiments, pharmaceutical compositions described herein are administered intermittently (e.g. drug holiday that includes a period of time in which the formulation is not administered or is administered in a reduced amount).

The amount of a given agent that will correspond to such an amount will vary depending upon factors such as the particular compound, disease or condition and its severity, and the identity (e.g., weight) of the subject or host in need of treatment, but can nevertheless be determined in a manner recognized in the field according to the particular circumstances surrounding the case, including, e.g., the specific agent being administered, the condition being treated, and the subject or host being treated. In general, however, doses employed for adult human treatment will typically be in the range from about 0.02 to about 5000 mg per day, in some embodiments, from about 1 to about 1500 mg per day. The desired dose may conveniently be presented in a single dose or as divided doses administered simultaneously (or over a short period of time) or at appropriate intervals, for example as two, three, four, or more sub-doses per day.

### Certain Terminology

Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein and in the appended claims, the singular forms “a”, “an”, and “the” include plural reference unless the context clearly dictates otherwise. Thus, for example, reference to “an excipient” is a reference to one or more excipients and equivalents thereof known to those skilled in the art, and so forth.

The term “about” is used to indicate that a value includes the standard level of error for the device or method being employed to determine the value. In some embodiments, the level of error is 10%.

The use of the term “or” in the claims is used to mean “and/or” unless explicitly indicated to refer to alternatives only or the alternatives are mutually exclusive, although the disclosure supports a definition that refers to only alternatives and to “and/or.”

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has,” or “includes” one or more steps is not limited to possessing only those one or more steps and also covers other unlisted steps.

“Optional” or “optionally” may be taken to mean that the subsequently described structure, event, or circumstance may or may not occur, and that the description includes instances where the events occurs and instances where it does not.

As used herein, the term “therapeutic” means an agent utilized to treat, combat, ameliorate, prevent, or improve an unwanted condition or disease of a patient.

“Administering”, when used in conjunction with a therapeutic, means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with Compound A formulation, can include, but is not limited to, providing Compound A formulation into or onto the target tissue; providing Compound A formulation systemically to a patient by, e.g., oral administration whereby the therapeutic reaches the target tissue or cells. “Administering” a formulation may be accomplished by injection, topical administration, and oral administration or by other methods alone or in combination with other known techniques.

The term “animal” as used herein includes, but is not limited to, humans and non-human vertebrates such as wild, domestic, and farm animals. As used herein, the terms “patient,” “subject,” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. Other examples of subjects include experimental animals such as mice, rats, dogs, cats, goats, sheep, pigs, and cows.

By “pharmaceutically acceptable”, it is meant the carrier, diluent, or excipient must be compatible with the other ingredients of the formulation and not deleterious to the recipient thereof.

The term “pharmaceutical composition”, as used herein refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein, refers to the amount of active compound or pharmaceutical agent that elicits a biological or medicinal response in a tissue, system, animal, individual, or human that is being sought by a researcher, veterinarian, medical doctor, or other clinician, which includes one or more of the following: (1) preventing the disease; for example, preventing a disease, condition, or disorder in an individual that may be predisposed to the disease, condition, or disorder but does not yet experience or display the pathology or symptomatology of the disease, (2) inhibiting the disease; for example, inhibiting a disease, condition, or disorder in an individual that is experiencing or displaying the pathology or symptomatology of the disease, condition, or disorder (i.e., arresting further development of the pathology and/or symptomatology), and (3) ameliorating the disease; for example, ameliorating a disease, condition, or disorder in an individual that is experiencing or displaying the pathology or symptomatology of the disease, condition, or disorder (i.e., reversing the pathology and/or symptomatology).

The terms “treat,” “treated,” “treatment,” or “treating” as used herein, refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “carrier,” as used herein, refers to relatively nontoxic chemical compounds or agents that facilitate the incorporation of a compound into cells or tissues. In some embodiments, the carrier is an aqueous carrier.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to, a phosphate buffered saline solution.

The terms “accelerated conditions” include temperature and/or relative humidity (RH) that are above ambient levels (e.g. 25±3° C.; 55±10% RH). In some instances, an accelerated condition is at about 30° C., about 35° C., about 40° C., about 45° C., about 50° C., about 55° C., or about 60° C. In other instances, an accelerated condition is about 60% RH, about 65% RH, about 70% RH, about 75% RH, or about 80% RH. In further instances, an accelerated condition is about 40° C. or 60° C. at ambient humidity. In yet further instances, an accelerated condition is about 40° C. at 75±5% RH humidity.

All publications, patents, and patent applications mentioned in this specification are herein incorporated by reference to the same extent as if each individual publication, patent, or patent application was specifically and individually indicated to be incorporated by reference.

### EXAMPLES

### Example 1: Polymorph Screening of Freebase N-(5-(6-chloro-2,2-difluorobenzo[d][1,3]dioxol-5-yl)pyrazin-2-yl)-2-fluoro-6-methylbenzamide

### X-Ray Powder Diffraction (XRPD)

PANalytical Empyrean X-ray powder diffractometer (XRPD) with 12-well auto sample stage was used. Typical XRPD parameters used are listed in Table 1.

TABLE 1
Typical XRPD parameters

| Parameters | Reflection mode |
|---|---|
| X-Ray wavelength | Cu, kα, |
| | Kα1 (Å): 1.540598, |
| | Kα2 (Å): 1.544426 |
| | Kα2/Kα1 intensity ratio: 0.50 |
| Sample Stage | 12-well auto sample stage |
| X-Ray tube setting | 45 kV, 40 mA |
| Divergence slit | Automatic |
| Monochromator | None |
| Scan mode | Continuous |
| Scan range (°2TH) | 3°-40° |
| Step size (°2TH) | 0.0170 |
| Scan speed (°/min) | About 10 |

### Differential Scanning Calorimetry (DSC)

Instrument: TA Q200/2000 DSC from TA Instruments

Method: Ramp from RT to desired temperature at a heating rate of 10° C./min using N₂ as the purge gas, with pan crimped.

### Thermogravimetric Analysis (TGA)

Instrument: TA Q500/Q5000 TGA from TA Instruments

Method: Ramp from RT to desired temperature at a heating rate of 10° C./min using N₂ as the purge gas.

Different crystallization or solid transition methods were used in the polymorph screening to discover as many crystalline forms as possible. The methods utilized are summarized in Table 2, including slow evaporation, slow cooling, polymer induced crystallization, slurry conversion, anti-solvent addition, sonication induced crystallization and heat-cooling.

TABLE 2
Summary of polymorph screening

| | No. of | |
|---|---|---|
| Method | Experiments | Solid Form |
| Slow evaporation | 12 | Form A |
| Slow cooling | 18 | Form A |
| Polymer induced crystallization | 9 | Form A, Form B, |
| | | Form C |
| Slurry conversion | 34 | Form A, Form D |
| Anti-solvent addition | 16 | Form A |
| Sonication induced crystallization | 7 | Form A |
| Heat-cooling | 11 | Form A |
| In-depth slurry experiment | 22 | Form A |
| Total | 129 | Form A |

### Slow Evaporation

Slow evaporation experiments were performed in 12 different solvent systems. Approximately 8 mg of N-(5-(6-chloro-2,2-difluorobenzo[d][1,3]dioxol-5-yl)pyrazin-2-yl)-2-fluoro-6-methylbenzamide Form A was dissolved with 0.1-1.6 mL of solvent in each HPLC glass vial. The visually clear solutions were subjected to slow evaporation at ambient temperature to dryness. The solids obtained were isolated for XRPD analysis. Results summarized in Table 3 indicate that only Form A was obtained.

TABLE 3
Summary of slow evaporation experiments

| Solvent, v/v | Solid Form |
|---|---|
| EtOH | Form A |
| EtOH/H₂O (19/1, v/v) | Form A |
| Acetonitrile | Form A |
| Acetonitrile/H₂O (19/1, v/v) | Form A |
| Acetone | Form A |
| Acetone/H₂O (19/1, v/v) | Form A |
| THF | Form A |
| THF/H₂O (19/1, v/v) | Form A |
| 1,4-Dioxane | Form A |
| 1,4-Dioxane/H₂O (19/1, v/v) | Form A |
| IPA | Form A |
| IPA/H₂O (19/1, v/v) | Form A |

### Slow Cooling

Slow cooling experiments were performed in 18 different solvent systems. Approximately 8 mg of N-(5-(6-chloro-2,2-difluorobenzo[d][1,3]dioxol-5-yl)pyrazin-2-yl)-2-fluoro-6-methylbenzamide Form A was suspended in 0.5 mL of corresponding solvent at 50° C. and equilibrated for 0.5 hr. Suspensions obtained were then filtered with syringe and Nylon membrane (pore size of 0.45 μm) at 50° C. The filtrates were collected and cooled from 50° C. to 5° C. at a rate of 0.1° C./min. If no precipitation was observed, the solution was evaporated at ambient temperature to induce precipitation. The solids were isolated for XRPD analysis and results summarized in Table 4 indicate that only Form A was obtained.

TABLE 4
Summary of slow cooling experiments

| | Water | Observation | |
|---|---|---|---|
| Solvent, v/v | activity | (5° C.) | Solid Form |
| MeOH/H₂O (1/1, v/v) | 0.76 | clear | Form A* |
| Acetonitrile/H₂O (1/1, v/v) | 0.85 | clear | Form A* |
| THF/H₂O (1/1, v/v) | 0.99 | clear | Form A* |
| 1,4-Dioxane/H₂O (1/1, v/v) | 0.98 | clear | Form A* |
| NMP/H₂O (1/1, v/v) | 0.83 | clear | Form A* |
| Acetone | 0.00 | clear | Form A* |
| Acetone/H₂O (0.98/0.02, v/v) | 0.25 | clear | Form A* |
| Acetone/H₂O (0.95/0.05, v/v) | 0.40 | clear | Form A* |
| Acetone/H₂O (0.85/0.15, v/v) | 0.61 | clear | Form A* |
| Acetone/H₂O (0.60/0.40, v/v) | 0.80 | clear | Form A* |
| THF | 0.00 | clear | Form A* |
| THF/H₂O (0.98/0.02, v/v) | 0.21 | clear | Form A* |
| THF/H₂O (0.95/0.05, v/v) | 0.45 | clear | Form A* |
| THF/H₂O (0.92/0.08, v/v) | 0.62 | clear | Form A* |
| THF/H₂O (0.87/0.13, v/v) | 0.80 | clear | Form A* |
| MeOH/Acetone/H₂O | — | clear | Form A* |
| (1/1/1, v/v/v) | | | |
| IPA/THF/H₂O | — | precipitation | Form A |
| (1/1/1, v/v/v) | | | |
| DMSO/1,4-Dioxane/H₂O | — | clear | Form A* |
| (1/1/1, v/v/v) | | | |

*The solid was obtained from slow evaporation

### Polymer Induced Crystallization

Polymer induced crystallization experiments were performed in 9 different solvent systems. Approximately 8 mg of N-(5-(6-chloro-2,2-difluorobenzo[d][1,3]dioxol-5-yl)pyrazin-2-yl)-2-fluoro-6-methylbenzamide Form A was dissolved in 0.1-1.5 mL of solvent in each HPLC glass vial. Approximately 1.0 mg of polymer (mixtures of six polymers including PVA, PVC, PVAC, PVP, HPMC and MC at the mass ratio of 1.0) was added into the visually clear solutions. All the samples were evaporated slowly at ambient temperature to dryness. The solids obtained were isolated for XRPD analysis. Results summarized in Table 5, below, indicate that Form A and two potentially new crystalline forms (Form B and Form C) were obtained.

TABLE 5
Summary of polymer induced crystallization experiments

| Solvent, v/v | Polymer | Solid Form |
|---|---|---|
| EtOH/H₂O (19/1, v/v) | mixed polymer | Form B |
| Acetonitrile/H₂O(19/1, v/v) | (PVAC, HPMC, PVC, | Form A |
| Acetone/H₂O (19/1, v/v) | MC, PVP, PVA) | Form A |
| THF/H₂O (19/1, v/v) | 1:1:1:1:1:1 | Form A |
| 1,4-Dioxane/H₂O (19/1, v/v) | | Form A |
| IPA/H₂O (19/1, v/v) | | Form A |
| MeOH/Acetone/H₂O | | Form C^{#} |
| (1/1/1, v/v/v) | | |
| IPA/THF/H₂O | | Form A |
| (1/1/1, v/v/v) | | |
| DMSO/1,4-Dioxane/H₂O | | Form A |
| (1/1/1, v/v/v) | | |

PVP: Polyvinyl pyrrolidone,
HPMC: Hypromellose
PVC: Polyvinylchloride,
PVA: polyvinyl alcohol
PVAC: polyvinyl acetate,
MC: methyl cellulose
*Filter the suspension with syringe and Nylon membrane (pore size of 0.45 μm), and evaporate the filtrate.

### Slurry Conversion

Slurry conversion experiments were conducted under 34 conditions. Approximately 8 mg of N-(5-(6-chloro-2,2-difluorobenzo[d][1,3]dioxol-5-yl)pyrazin-2-yl)-2-fluoro-6-methylbenzamide Form A was suspended in 0.5 mL of each solvent. After the suspensions were stirred at ambient temperature or 50° C. for three days, the solids were isolated for XRPD analysis. If the suspensions turned into clear solutions upon slurry, the clear solutions were subjected to slow evaporation at ambient temperature. Results summarized in Table 6 and Table 7 indicate that Form A and a potentially new crystalline form Form D were obtained.

TABLE 6
Summary of slurry conversion experiments at ambient temperature

| | Water | | |
|---|---|---|---|
| Solvent, v/v | activity | Temperature | Solid Form |
| MeOH/H₂O (1/19, v/v) | 0.98 | RT | Form A |
| Acetonitrile/H₂O (1/19, v/v) | 0.98 | RT | Form A |
| Acetone/H₂O (1/19, v/v) | 0.99 | RT | Form A |
| THF/H₂O (1/19, v/v) | 0.99 | RT | Form A |
| 1,4-Dioxane/H₂O (1/19, v/v) | 0.99 | RT | Form A |
| DMSO/H₂O (1/19, v/v) | 0.99 | RT | Form A |
| IPA | 0.00 | RT | Form A |
| IPA/H₂O (0.98/0.02, v/v) | 0.22 | RT | Form A |
| IPA/H₂O (0.95/0.05, v/v) | 0.44 | RT | Form A |
| IPA/H₂O (0.92/0.08, v/v) | 0.59 | RT | Form A |
| IPA/H₂O (0.85/0.15, v/v) | 0.80 | RT | Form A |
| H₂O | 1.00 | RT | Form A |
| EtOH | 0.00 | RT | Form A* |
| EtOH/H₂O(0.97/0.03, v/v) | 0.20 | RT | Form A* |
| EtOH/H₂O (0.93/0.07, v/v) | 0.39 | RT | Form A |
| EtOH/H₂O (0.85/0.15, v/v) | 0.62 | RT | Form A |
| EtOH/H₂O (0.70/0.30, v/v) | 0.81 | RT | Form A |

*The solid was obtained from slow evaporation

TABLE 7
Summary of slurry conversion experiments at 50° C.

| | Water | Temperature | |
|---|---|---|---|
| Solvent, v/v | activity | (° C.) | Solid Form |
| MeOH/H₂O (1/19, v/v) | 0.98 | 50 | Form A |
| Acetonitrile/H₂O (1/19, | 0.98 | 50 | Form A |
| v/v) | | | |
| Acetone/H₂O (1/19, v/v) | 0.99 | 50 | Form A |
| THF/H₂O (1/19, v/v) | 0.99 | 50 | Form A |
| 1,4-Dioxane/H₂O (1/19, | 0.99 | 50 | Form A |
| v/v) | | | |
| DMSO/H₂O (1/19, v/v) | 0.99 | 50 | Form A |
| IPA | 0.00 | 50 | Form A* |
| IPA/H₂O (0.98/0.02, v/v) | 0.22 | 50 | Form A* |
| IPA/H₂O (0.95/0.05, v/v) | 0.44 | 50 | Form A* |
| IPA/H₂O (0.92/0.08, v/v) | 0.59 | 50 | Form A* |
| IPA/H₂O (0.85/0.15, v/v) | 0.80 | 50 | Form A |
| H₂O | 1.00 | 50 | Form A |
| EtOH | 0.00 | 50 | Form A* |
| EtOH/H₂O(0.97/0.03, | 0.20 | 50 | Form A* |
| v/v) | | | |
| EtOH/H₂O (0.93/0.07, | 0.39 | 50 | Form A* |
| v/v) | | | |
| EtOH/H₂O (0.85/0.15, | 0.62 | 50 | Form D^{#}* |
| v/v) | | | |
| EtOH/H₂O (0.70/0.30, | 0.81 | 50 | Form A |
| v/v) | | | |

^{#}Potentially new crystalline form
*The solid was obtained from slow evaporation

### Anti-Solvent Addition

The anti-solvent addition experiments were conducted under 16 conditions. Approximately 15 mg of N-(5-(6-chloro-2,2-difluorobenzo[d][1,3]dioxol-5-yl)pyrazin-2-yl)-2-fluoro-6-methylbenzamide Form A was dissolved in 0.1-3.0 mL of each solvent to get a clear solution. 3.0-18.0 mL of each anti-solvent was added drop-wise into above clear solution at ambient temperature. The precipitate was isolated for XRPD analysis. Slow evaporation experiments were conducted for clear solutions. The results summarized in Table 8 suggest that Form A was obtained.

TABLE 8
Summary of anti-solvent addition experiments
at ambient temperature

| | Anti- | | |
|---|---|---|---|
| Solvent | solvent | Observation | Solid Form |
| IPA | H₂O | precipitation | Form A |
| Acetonitrile | H₂O | precipitation | Form A |
| Acetone | H₂O | precipitation | Form A |
| 2-MeTHF | H₂O | precipitation | Form A |
| 1,4-Dioxane | H₂O | precipitation | Form A |
| DMAc | H₂O | precipitation | Form A |
| MeOH/Acetonitrile (1/1, v/v) | H₂O | precipitation | Form A |
| EtOH/DMSO (1/1, v/v) | H₂O | precipitation | Form A |
| THF/IPA (1/1, v/v) | H₂O | precipitation | Form A |
| Acetonitrile/2-MeTHF (1/1, v/v) | H₂O | precipitation | Form A |
| Acetonitrile/NMP (1/1, v/v) | H₂O | precipitation | Form A |
| Acetone/DMAc (1/1, v/v) | H₂O | precipitation | Form A |
| Acetone/1,4-Dioxane (1/1, v/v) | H₂O | precipitation | Form A |
| THF/DMSO (1/1, v/v) | H₂O | precipitation | Form A |
| THF/1,4-Dioxane (1/1, v/v) | H₂O | precipitation | Form A |
| NMP/1,4-Dioxane (1/1, v/v) | H₂O | precipitation | Form A |

### Sonication Induced Crystallization

Sonication induced crystallization experiments were performed in 7 different solvent systems. Approximately 15 mg of N-(5-(6-chloro-2,2-difluorobenzo[d][1,3]dioxol-5-yl)pyrazin-2-yl)-2-fluoro-6-methylbenzamide was suspended in 0.3 mL of solvent in each HPLC glass vial. All samples were sonicated for 0.5 h at ambient temperature. The solids obtained were isolated for XRPD analysis. Results summarized in Table 9, below, indicate that Form A was obtained.

TABLE 9
Summary of sonication induced crystallization experiments

| Solvent, v/v | Temperature | Solid Form |
|---|---|---|
| MeOH/H₂O (1/19, v/v) | RT | Form A |
| Acetonitrile/H₂O (1/19, v/v) | RT | Form A |
| Acetone/H₂O (1/19, v/v) | RT | Form A |
| THF/H₂O (1/19, v/v) | RT | Form A |
| 1,4-Dioxane/H₂O (1/19, v/v) | RT | Form A |
| DMSO/H₂O (1/19, v/v) | RT | Form A |
| H₂O | RT | Form A |

### Heat-Cooling

Heat-cooling experiments were performed in 11 different solvent systems. Approximately 15 mg of N-(5-(6-chloro-2,2-difluorobenzo[d][1,3]dioxol-5-yl)pyrazin-2-yl)-2-fluoro-6-methylbenzamide Form A was suspended in 0.5 mL of solvent. The samples were kept into a temperature-controlled biochemical incubator and slurried at a rate of 1000 r/min on a magnetic stirrer for about 9 hours. The heat-cooling cycle was programmed as following: 1) Ramp to 50° C. in 30 min, and equilibrate at 50° C. for about 30 min; 2) Cool to 5° C. in 450 min, and equilibrate at 5° C. for about 30 min; 3) Repeat the heat-cooling cycle three times before analyzing the precipitate. Slow evaporation experiments were conducted for the clear solutions. The results summarized in Table 10, below, suggest that Form A was obtained.

TABLE 10
Summary of heat-cooling experiments

| Solvent, v/v | Solid Form |
|---|---|
| EtOH/Heptane, 1/19 | Form A |
| IPA/Heptane, 1/19 | Form A |
| Acetone/Heptane, 1/19 | Form A |
| MIBK/Heptane, 1/19 | Form A |
| IPAc/Heptane, 1/19 | Form A |
| MTBE/Heptane, 1/19 | Form A |
| THF/Heptane, 1/19 | Form A |
| 1,4-Dioxane/Heptane, 1/19 | Form A |
| NMP/Heptane, 1/19 | Form A |
| DCM/Heptane, 1/19 | Form A* |
| Toluene/Heptane, 1/19 | Form A |

*The solid was obtained from slow evaporation

### In-Depth Slurry Experiments

In-depth slurry experiments were conducted in 22 conditions at various water activities. Approximately 20 mg of N-(5-(6-chloro-2,2-difluorobenzo[d][1,3]dioxol-5-yl)pyrazin-2-yl)-2-fluoro-6-methylbenzamide was suspended in 0.5 mL of each solvent. After the suspensions were stirred at ambient temperature or 50° C. for 22 days, the solids were isolated for XRPD analysis. Results summarized in Table 11 and Table 12 indicate that only Form A was obtained.

TABLE 11
Summary of in-depth slurry experiments at RT

| | Water | | |
|---|---|---|---|
| Solvent, v/v | activity | Observation | Solid Form |
| MeOH | 0.00 | clear solution | N/A |
| MeOH/H₂O (0.94:0.06, v/v) | 0.19 | suspension | Form A |
| MeOH/H₂O (0.84:0.16, v/v) | 0.40 | suspension | Form A |
| MeOH/H₂O (0.69:0.31, v/v) | 0.60 | suspension | Form A |
| MeOH/H₂O (0.42:0.58, v/v) | 0.80 | suspension | Form A |
| H₂O | 1.00 | suspension | Form A |
| EtOH | 0.00 | suspension | Form A |
| EtOH/H₂O (0.97:0.03, v/v) | 0.20 | suspension | Form A |
| EtOH/H₂O (0.93:0.07, v/v) | 0.39 | suspension | Form A |
| EtOH/H₂O (0.85:0.15, v/v) | 0.62 | suspension | Form A |
| EtOH/H₂O (0.70:0.30, v/v) | 0.81 | suspension | Form A |

N/A: not applicable.

TABLE 12
Summary of in-depth slurry experiment at 50° C.

| | Water | | |
|---|---|---|---|
| Solvent, v/v | activity | Observation | Solid Form |
| MeOH | 0.00 | clear solution | N/A |
| MeOH/H₂O (0.94:0.06, v/v) | 0.19 | clear solution | N/A |
| MeOH/H₂O (0.84:0.16, v/v) | 0.40 | suspension | Form A |
| MeOH/H₂O (0.69:0.31, v/v) | 0.60 | suspension | Form A |
| MeOH/H₂O (0.42:0.58, v/v) | 0.80 | suspension | Form A |
| H₂O | 1.00 | suspension | Form A |
| EtOH | 0.00 | clear solution | N/A |
| EtOH/H₂O (0.97:0.03, v/v) | 0.20 | clear solution | N/A |
| EtOH/H₂O (0.93:0.07, v/v) | 0.39 | suspension | Form A |
| EtOH/H₂O (0.85:0.15, v/v) | 0.62 | suspension | Form A |
| EtOH/H₂O (0.70:0.30, v/v) | 0.81 | suspension | Form A |

N/A: not applicable.

### Example 1A: Characterization of New Crystalline Forms

Four crystalline forms (Form A, Form B, Form C and Form D) were obtained as summarized in Table 13.

TABLE 13
Summary of crystalline forms

| Crystalline | | DSC endotherms |
|---|---|---|
| Hits | Crystallinity | (° C., onset) |
| Form A | High | 156.6 |
| Form B | High | 54.3, 155.9 |
| Form C | High | 82.4, 104.6 155.9 |
| Form D | High | 100.5, 155.7 |

### Characterization of Form A

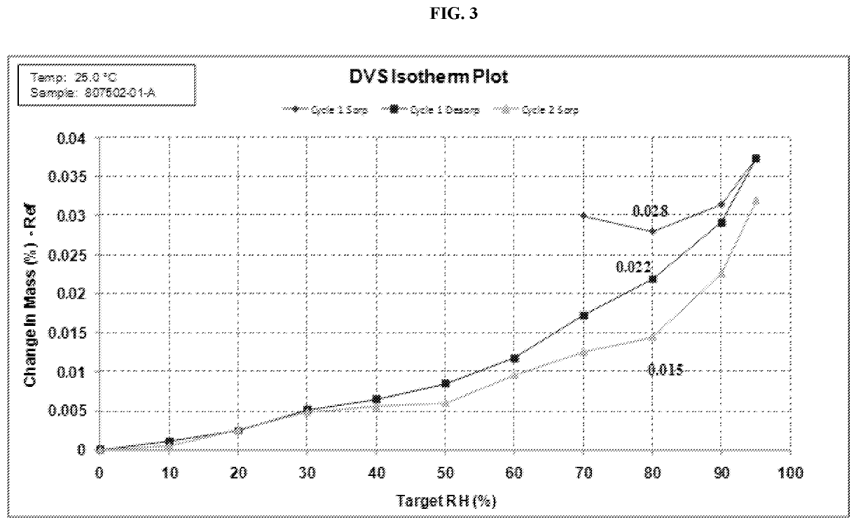

The XRPD pattern shown in FIG. 1 indicates that N-(5-(6-chloro-2,2-difluorobenzo[d][1,3]dioxol-5-yl)pyrazin-2-yl)-2-fluoro-6-methylbenzamide freebase Form A is highly crystalline. Differential Scanning Calorimetry (DSC) and Thermogravimetric Analysis (TGA) curves exhibit a sharp melting point of 156.6° C. (onset temperature) and a weight loss of 1.0% up to 150° C., respectively, as displayed in FIG. 2. The DVS isotherm plot in FIG. 3 shows that Form A is not hygroscopic, with a water uptake level of <0.03% at 80% RH. The crystal size of Form A is in the range of ˜few μm to about 50 μm.

Solubility of N-(5-(6-chloro-2,2-difluorobenzo[d][1,3]dioxol-5-yl)pyrazin-2-yl)-2-fluoro-6-methylbenzamide Form A

The Solubility of N-(5-(6-chloro-2,2-difluorobenzo[d][1,3]dioxol-5-yl)pyrazin-2-yl)-2-fluoro-6-methylbenzamide freebase Form A was determined in 20 solvents at RT. These experiments were conducted by adding approximately 2 mg of sample into a 3-mL glass vial. Solvents in Table 14 were then added in 50 μL increments into the vials until the solids were dissolved or a total volume of 2 mL was reached. The solubility estimation was used to guide the solvent selection in polymorph screening. N-(5-(6-chloro-2,2-difluorobenzo[d][1,3]dioxol-5-yl)pyrazin-2-yl)-2-fluoro-6-methylbenzamide freebase Form A is soluble in MeOH, Acetic acid, Acetonitrile, Acetone, MIBK, EtOAc, IPAc, MTBE, THF, 2-MeTHF, 1,4-Dioxane, NMP, DMSO, DCM, Toluene and DMAc (>18.0 mg/mL), while it is insoluble in Heptane and H₂O (<1.3 mg/mL).

TABLE 14
Solubility of N-(5-(6-chloro-2,2-difluorobenzo[d][1,3]dioxol-
5-yl)pyrazin-2-yl)-2-fluoro-6-methylbenzamide freebase Form A

| | Solubility | | Solubility |
|---|---|---|---|
| Solvent | (mg/mL) | Solvent | (mg/mL) |
| MeOH | >18.0 | THF | >20.0 |
| EtOH | 10.0 < S < 20.0 | 2-MeTHF | >26.0 |
| IPA | 9.0 < S < 18.0 | 1,4-Dioxane | >24.0 |
| Acetic acid | >22.0 | NMP | >26.0 |
| Acetonitrile | >22.0 | DMSO | >20.0 |
| Acetone | >22.0 | DCM | >22.0 |
| MIBK | >22.0 | Toluene | >22.0 |
| EtOAc | >22.0 | Heptane | <1.3 |
| IPAc | >24.0 | DMAc | >22.0 |
| MTBE | >18.0 | H₂O | <1.0 |

IPA: Isopropyl alcohol
MIBK: Methyl isobutyl ketone
EtOAc: Ethyl acetate
IPAc: Isopropyl acetate
MTBE: Methyl tert-butyl ether
THF: Tetrahydrofuran
NMP: N-methyl-2-pyrrolidone
DMSO: Dimethyl sulfoxide
DCM: Dichloromethane
DMAc: Dimethylacetamide

### Characterization of Form B

Form B was obtained from polymer induced crystallization in EtOH/H₂O (19/1, v/v). The XRPD pattern of Form B in FIG. 4 shows minor differences compared to Form A. The DSC curve of Form B (FIG. 5) exhibits an endotherm at 54.3° C. (onset temperature) attributed to dehydration/desolvation before melting at 155.9° C. (onset temperature).

### Characterization of Form C

Form C was obtained from polymer induced crystallization in MeOH/Acetone/H₂O (1/1/1, v/v/v). The XRPD pattern of Form C in FIG. 6 shows minor differences compared to Form A. The DSC curve of Form C in FIG. 7 exhibits two endotherms at 82.4° C. and 104.6° C. (peak temperature), attributed to dehydration/desolvation before melting at 155.9° C. (onset temperature).

### Characterization of Form D

Form D was obtained from solution evaporation after slurrying in EtOH/H₂O (0.85/0.15, v/v) at 50° C. for 3 days. The XRPD pattern of Form D in FIG. 8 shows minor differences compared to Form A. The DSC curve of Form D in FIG. 9 exhibits an endotherm at 100.5° C. (onset temperature), attributed to dehydration/desolvation before melting at 155.9° C. (onset temperature).

### Example 2: Initial Suspension Formulations

Ten compositions were prepared using various template compositions, containing lecithin, soybean oil (SBO) or medium chain triglycerides (MCT), glycerin or sucrose (non-ionic tonicity agent), edetate disodium di-hydrate (EDTA, chelating agent) in deionized water. Compound A (Form A) was added and agitated to reach solubility equilibrium at ambient room temperature. Each formulation was prepared in the following steps: Compound A (5 mg) was dispersed in each template vehicle. The formulation were then homogenized and at room temperature for >24 h and then passed sample through 0.45 m filter for analysis (HPLC).

The study compositions and analysis are tabulated in Table 15:

TABLE 15

| Formula (%, w/w) | F-1 | F-2 | F-3 | F-4 | F-5 | F-6 | F-7 | F-8 | F-9 | F-10 |
|---|---|---|---|---|---|---|---|---|---|---|
| Compound A | * | * | * | * | * | * | * | * | * | * |
| Egg Lecithin | 5 | 10 | 15 | 20 | 5 | 3 | | | 10 | 5 |
| Soy Lecithin | | | | | | | 4 | 10 | | |
| SBO | 5 | 5 | 5 | 5 | 10 | 2.5 | 1 | 4 | | 5 |
| MCT | | | | | | | | 4 | 5 | 5 |
| Cholesterol | | | | | | | | 0.6 | | |
| VES | | | | | | | | 0.3 | | |
| Sucrose | 8.2 | 8.2 | 8.2 | 8.2 | 8.2 | | | 17.5 | 5 | 10 |
| Glycerin | | | | | | 2.25 | 2.25 | | | |
| EDTA | 0.0055 | 0.0055 | 0.0055 | 0.0055 | 0.0055 | 0.0055 | 0.0055 | 0.0055 | 0.0055 | 0.005 |
| WFI (q.s.) | 100 | 100 | 100 | 100 | 100 | 100 | 100 | 100 | 100 | 100 |
| Results | | | | | | | | | | |
| Assay (mg/mL) | 1.6 | 3 | 3.4 | 4 | 1.8 | 0.6 | 0.5 | 3.6 | 2.5 | 2.3 |
| Appearance | O | T | T, V | T, V | O | T | T | O, V | T | O |

* Excess of Compound A was added to maintain saturation in vehicle
VES = vitamin E succinate, USP
T: Translucent
O: Opaque
V: Viscous

Conclusion: Compound A solubility was >2.4 mg/mL in the emulsion composition containing >10% egg lecithin. Emulsion composition was viscous when lecithin>15%.

### Example 3: Stability of Formulation F-9 (Small Scale)

The formulation was prepared at about 1.2 g scale. The composition is tabulated in table 16.

TABLE 16

| Formula (%, w/w) | F-9 |
|---|---|
| Compound A | 0.24 |
| Egg Lecithin | 10 |
| MCT | 5 |
| Sucrose | 5 |
| EDTA | 0.0055 |
| NaOH/HCl | Adjust pH to neutral |
| SWFI (q.s.) | 100 |

Procedures: Compound A (Form A) was added to F-9 vehicle containing MCT/Egg/lecithin/Sucrose/EDTA in a plastic tube. The formulation was mixed until uniform and complete drug dissolution. The emulsion was sterilized through 0.2 μm membrane filter. The samples were placed at 2-8° C. and 25° C. for stability evaluation for 2 weeks. The samples were tested for appearance, pH, Compound A assay and purity by HPLC, mean droplet size and globule size distribution in lipid injectable emulsions (USP<729>) and the results are shown in Table 17.

TABLE 17

| | | | Mean | | | |
|---|---|---|---|---|---|---|
| | | | Droplet Size | PFAT5 | Assay | Purity |
| Stability | Appearance | pH | (nm) | (%) | (mg/mL) | (%) |
| 2-8° C., initial | OWT | n/a | n/a | n/a | n/a | n/a |
| 2-8° C., 2 weeks | OWT | 7.4 | n/a | n/a | 2.38 | 99.9 |
| 25° C., 2 weeks | OWT | 7.1 | n/a | n/a | 2.38 | 99.9 |

OWT: Off-white translucent emulsion
n/a = not performed

Conclusion: Compound A remained unchanged in appearance and HPLC assay after 2 weeks at 2-8° C. and 25° C.

### Example 4: Stability of Formulation F-9 (Large Scale)

The formulation was prepared at about 100 g scale. The composition is tabulated in table 18.

TABLE 18

| Formula (%, w/w) | F-9A |
|---|---|
| Compound A (Form A) | 0.2 |
| Egg Lecithin | 10 |
| MCT | 5 |
| Sucrose | 5 |
| EDTA | 0.0055 |
| NaOH/HCl | Adjust pH to neutral |
| SWFI (q.s.) | 100 |

Procedures: Compound A (Form A) was added to egg lecithin, MCT, EDTA, sucrose and SWFI in a vessel. The mixture was mixed until uniform and the pH was adjusted pH to ˜8 with NaOH/HCl. The coarse emulsion was homogenized at high pressure until droplet size <120 nm and then sterilized through 0.2 μm membrane filter. The final emulsion was filled in sterile glass vials and closed with serum stopper and crimp-sealed for stability evaluation at 2-8° C. and 25° C. with sampling at 0, 15 and 30 days. The emulsion was tested for appearance, pH, Compound A assay and purity by HPLC, mean droplet size and globule size distribution in lipid injectable emulsions (USP<729>) and the results are compiled in Table 19.

TABLE 19

| | | | Mean | | | | |
|---|---|---|---|---|---|---|---|
| | | | Droplet Size | PFAT5 | Assay | % | Purity |
| Stability | Appearance | pH | (nm) | (%) | (mg/mL) | Initial | (%) |
| 2-8° C., initial | OWT | 7.5 | 54 | 0.002 | 2.12 | 100 | 99.9 |
| 2-8° C., 4 weeks | OWT | n/a | n/a | n/a | n/a | n/a | n/a |
| 25° C., 4 weeks | OWT | n/a | n/a | n/a | n/a | n/a | n/a |
| 2-8° C., 3 Mo | OWT | 6.0 | 56 | 0.002 | 2.17 | 102 | 99.6 |
| 2-8° C., 6 Mo | OWT | 5.7 | 57 | 0.002 | 2.09 | 99 | 99.8 |
| 25° C., 3 Mo | OWT | 5.1 | 69 | 0.002 | 2.10 | 99 | 99.6 |

OWT: Off-white translucent emulsion
n/a = not performed

Conclusion: The 0.2% Compound A emulsion (F-9A) remained unchanged in appearance and HPLC assay, PFAT5 and mean droplet size after 3 Mo at 2-8° C. and 25° C. The analysis of the fat globule-size distribution, PFAT5, USP <729> method II, was used to assess the emulsion physical stability. The PFAT5 acceptance criteria was not more than 0.05%.

### Example 5: Emulsions Optimization

Over 36 emulsions compositions were prepared to rationally define the optimal oil, phospholipid, concentration, ratio, pH, . . . for Compound A≥2.5 mg/mL formulation. The formulations were prepared containing Compound A (Form A), egg lecithin (E-80), medium chain triglycerides (MCT), Glycerin USP, edetate disodium di-hydrate USP (EDTA), NaOH (as pH adjustor), and sterile water for injection USP (SWFI), according to the compositions tabulated in Tables 20-25. The aqueous phase pH was adjusted to 8 by the diluted NaOH solution.

TABLE 20

| Formula (%) | F-30 | F-31 | F-32 | F-33 | F-34 | F-35 | F-36 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 0 | 0 | 0 | 0 | 0 | 0 | 0 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

TABLE 21

| Formula (%) | F-37 | F-38 | F-39 | F-40 | F-41 | F-42 | F-43 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 1 | 1 | 1 | 1 | 1 | 1 | 1 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

TABLE 22

| Formula (%) | F-46 | F-47 | F-48 | F-49 | F-50r | F-51 | F-52 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 2.5 | 2.5 | 2.5 | 2.5 | 2.5 | 2.5 | 2.5 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

TABLE 23

| Formula (%) | F-53 | F-54 | F-55 | F-56 | F-57 | F-58 | F-59 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 5 | 5 | 5 | 5 | 5 | 5 | 5 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

TABLE 24

| Formula (%) | F-60 | F-61 | F-62 | F-63 | F-64 | F-65 | F-66 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 7.5 | 7.5 | 7.5 | 7.5 | 7.5 | 7.5 | 7.5 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

TABLE 25

| Formula (%) | F-67 | F-68 | F-69 | F-70 | F-71 | F-72 | F-73 |
|---|---|---|---|---|---|---|---|
| Compound A | 0.3 | 0.3 | 0.3 | 0.3 | 0.3 | 0.3 | 0.3 |
| E-80 | 1 | 2.5 | 5 | 7.5 | 10 | 12.5 | 15 |
| MCT | 10 | 10 | 10 | 10 | 10 | 10 | 10 |
| Glycerin | 2.25 | 2.25 | 2.25 | 2.25 | 2.25 | 2.25 | 2.25 |
| EDTA | 0.0055 | 0.0055 | 0.0055 | 0.0055 | 0.0055 | 0.0055 | 0.0055 |
| SWFI (q.s.) | 100 | 100 | 100 | 100 | 100 | 100 | 100 |

### Acceptance Criteria:

No less than 2.5 mg/mL Compound A
Mean oil droplet (Z-Ave, nm) size less than 150 nm
Pass through 0.2 μm sterile filtration
Meet droplet size distribution specification USP <729>, i.e. PFAT5 NMT 0.05%
Neutral pH (Range: 4-8)
Isotonic (Range: 240-350 mOsm/Kg)
Accelerated and long-term stability (>1 Mo at 2-8 and 25° C.)

### Procedure:

Prepared all vehicles by mixing lipid and aqueous phase ingredients and homogenized.
Added Compound A (Form A) at 0.3% concentration in each vehicle.
Homogenized and mixed overnight until uniform or achieved equilibrium.
Filtered emulsion through 0.45 μm Nylon membrane filter.
Evaluated appearance, drug concentration by HPLC assay, average droplet size and PFAT5.
Selected the top 5-10 formulations achieving acceptance criteria initially.
Placed at 40° C. for up to 2 weeks to monitor emulsion stability.
Selected the top 3-5 formulations meeting the proposed requirements after 1-2 weeks at 40° C.

### Methods:

Appearance: Record visual observation
Z-ave (nm): Measure mean oil droplet size by ZetaSizer (Malvern Instrument). Dilute 50 μL sample with 950 μL DI water at room temperature
Assay (%): Use the current HPLC method
Spin-X: Pass 0.5 mL emulsion through CoStar® Spin-X 0.2 μm Nylon filter (0.7 cm² surface area) at 3,144 G-force centrifuge for 60 sec at ambient room temperature. Evaluate completeness of emulsion passing through the filter.

### Results:

T: Translucent off-white to yellowish emulsion
O: Opaque off-white to yellowish emulsion
PPT: Drug precipitation

Time-0 Test Results are shown in Tables 26-31.

TABLE 26

| Formulation | | F-30 | F-31 | F-32 | F-33 | F-34 | F-35 | F-36 |
|---|---|---|---|---|---|---|---|---|
| E-80 (%) | | 1 | 2.5 | 5 | 7.5 | 10 | 12.5 | 15 |
| MCT (%) | | 0 | 0 | 0 | 0 | 0 | 0 | 0 |
| Test Results | | | | | | | | |
| Appearance | | T | T | T | T | T | T | T |
| Assay (%) | | 0.01 | 0.02 | 0.04 | 0.07 | 0.10 | 0.12 | 0.14 |
| Z-Ave (nm) | | 104 | 86 | 123 | 91 | 77 | 92 | 142 |
| 0.2 μm Spin-X | | n/a | n/a | n/a | n/a | n/a | n/a | n/a |
| Ratio | API:E- | 0.01 | 0.01 | 0.01 | 0.01 | 0.01 | 0.01 | 0.01 |
| | 80 | | | | | | | |
| | MCT:E- | 0.0 | 0.0 | 0.0 | 0.0 | 0.0 | 0.0 | 0.0 |
| | 80 | | | | | | | |

TABLE 27

| Formulation | | F-37 | F-38 | F-39 | F-40 | F-41 | F-42 | F-43 |
|---|---|---|---|---|---|---|---|---|
| E-80 (%) | | 1 | 2.5 | 5 | 7.5 | 10 | 12.5 | 15 |
| MCT (%) | | 1 | 1 | 1 | 1 | 1 | 1 | 1 |
| Test Results | | | | | | | | |
| Appearance | | T | T | T | T | T | T | T |
| Assay (%) | | 0.04 | 0.05 | 0.07 | 0.09 | 0.11 | 0.14 | 0.17 |
| Z-Ave (nm) | | 123 | 105 | 73 | 76 | 75 | 70 | 72 |
| 0.2 μm Spin-X | | n/a | n/a | n/a | n/a | n/a | n/a | n/a |
| Ratio | API:E-80 | 0.04 | 0.02 | 0.01 | 0.01 | 0.01 | 0.01 | 0.01 |
| | MCT:E-80 | 1.00 | 0.40 | 0.20 | 0.13 | 0.10 | 0.08 | 0.07 |

TABLE 28

| Formulation | | F-46 | F-47 | F-48 | F-49 | F-50 | F-51 | F-52 |
|---|---|---|---|---|---|---|---|---|
| E-80 (%) | | 1 | 2.5 | 5 | 7.5 | 10 | 12.5 | 15 |
| MCT (%) | | 2.5 | 2.5 | 2.5 | 2.5 | 2.5 | 2.5 | 2.5 |
| Test Results | | | | | | | | |
| Appearance | | O | T | T | T | T | T | T |
| Assay (%) | | 0.09 | 0.10 | 0.11 | 0.14 | 0.19 | 0.25 | 0.24 |
| Z-Ave (nm) | | 152 | 73 | 75 | 101 | 85 | 96 | 89 |
| 0.2 μm Spin-X | | n/a | n/a | n/a | n/a | Fail | Fail | n/a |
| Ratio | API:E-80 | 0.09 | 0.04 | 0.02 | 0.02 | 0.02 | 0.02 | 0.02 |
| | MCT:E-80 | 2.50 | 1.00 | 0.50 | 0.33 | 0.25 | 0.20 | 0.17 |

TABLE 29

| Formulation | | F-53 | F-54 | F-55 | F-56 | F-57 | F-58 | F-59 |
|---|---|---|---|---|---|---|---|---|
| E-80 (%) | | 1 | 2.5 | 5 | 7.5 | 10 | 12.5 | 15 |
| MCT (%) | | 5 | 5 | 5 | 5 | 5 | 5 | 5 |
| Test Results | | | | | | | | |
| Appearance | | O | O | T | T | T | T | T |
| Assay (%) | | 0.11 | 0.22 | 0.24 | 0.30 | 0.35 | 0.26 | 0.28 |
| Z-ave (nm) | | 159 | 133 | 103 | 88 | 96 | 79 | 80 |
| 0.2 μm Spin-X | | n/a | n/a | n/a | Pass | Pass | Fail | Fail |
| Ratio | API:E- | 0.11 | 0.09 | 0.05 | 0.04 | 0.04 | 0.02 | 0.02 |
| | 80 | | | | | | | |
| | MCT:E- | 5.00 | 2.00 | 1.00 | 0.67 | 0.50 | 0.40 | 0.30 |
| | 80 | | | | | | | |

TABLE 30

| Formulation | | F-60 | F-61 | F-62 | F-63 | F-64 | F-65 | F-66 |
|---|---|---|---|---|---|---|---|---|
| E-80 (%) | | 1 | 2.5 | 5 | 7.5 | 10 | 12.5 | 15 |
| MCT (%) | | 7.5 | 7.5 | 7.5 | 7.5 | 7.5 | 7.5 | 7.5 |
| Test Results | | | | | | | | |
| Appearance | | O | O | O | T | T | T | T |
| Assay (%) | | 0.08 | 0.18 | 0.23 | 0.26 | 0.35 | 0.33 | 0.38 |
| Z-ave (nm) | | 146 | 126 | 112 | 87 | 82 | 96 | 112 |
| 0.2 μm Spin-X | | n/a | n/a | n/a | Pass | Pass | Fail | Fail |
| Ratio | API:E-80 | 0.08 | 0.07 | 0.05 | 0.03 | 0.04 | 0.03 | 0.03 |
| | MCT:E-80 | 7.50 | 3.00 | 1.50 | 1.00 | 0.75 | 0.60 | 0.50 |

TABLE 31

| Formulation | | F-67 | F-68 | F-69 | F-70 | F-71 | F-72 | F-73 |
|---|---|---|---|---|---|---|---|---|
| E-80 (%) | | 1 | 2.5 | 5 | 7.5 | 10 | 12.5 | 15 |
| MCT (%) | | 10 | 10 | 10 | 10 | 10 | 10 | 10 |
| Test Results | | | | | | | | |
| Appearance | | O | O | O | O | T | T | T |
| Assay (%) | | 0.09 | 0.27 | 0.27 | 0.28 | 0.25 | 0.35 | 0.36 |
| Z-ave (nm) | | 177 | 150 | 120 | 151 | 90 | 100 | 86 |
| 0.2 μm Spin-X | | n/a | n/a | n/a | Fail | Pass | Fail | Fail |
| Ratio | API:E-80 | 0.09 | 0.11 | 0.05 | 0.04 | 0.03 | 0.03 | 0.02 |
| | MCT:E-80 | 10.00 | 4.00 | 2.00 | 1.33 | 1.00 | 0.80 | 0.67 |

Emulsion Stability for F-56, F-57, F58, F-63, F-64, F-65, and F-71 for 1 Week at 40° C. are shown in Tables 32.

TABLE 32

| Formulation | | F-56 | F-57 | F-58 | F-63 | F-64 | F-65 | F-71 |
|---|---|---|---|---|---|---|---|---|
| E-80 (%) | | 7.5 | 10 | 12.5 | 7.5 | 10 | 12.5 | 10 |
| MCT (%) | | 5 | 5 | 5 | 7.5 | 7.5 | 7.5 | 10 |
| Emulsion Stability | | | | | | | | |
| 2-8° C., Day 0 | Appearance | T | T | T | T | T | T | T |
| | Assay (%) | 0.30 | 0.35 | 0.26 | 0.26 | 0.35 | 0.33 | 0.25 |
| | Z-Ave (nm) | 88 | 96 | 79 | 87 | 82 | 96 | 90 |
| | PFAT5 (%) | 0.009 | 0.012 | 0.003 | 0.003 | 0.002 | 0.004 | 0.014 |
| 40° C., Day 8 | Appearance | O + PPT | 0 | 0 | 0 | 0 | 0 | 0 |
| | Assay (%) | 0.26 | 0.34 | 0.26 | 0.27 | 0.36 | 0.33 | 0.25 |
| | Z-Ave (nm) | 126 | 112 | 149 | 125 | 141 | 128 | 158 |
| | PFAT5 (%) | n/a | 0.043 | 0.054 | 0.093 | 0.012 | 0.037 | 0.060 |

Conclusion:
Compound A remained stable in F-57, F-58, F-63, F-64, F-65 and F-71 emulsions at 2-8° C. and after 8 days at 40° C. The HPLC assay data support drug concentration at >0.25% Compound A in formulation. The % purity remains unchanged at 99.9% on stability.
Drug precipitation was observed in F-56 after 8 days at 40° C. and failed to support a 0.25% emulsion.
The analysis of the fat globule-size distribution, PFAT5(%), was used to assess emulsion physical stability at 2-8° C. and 40° C. Three formulations, F-58, F-63 and F-71, after 8 days at 40° C., fails to meet USP <729> acceptance criteria, which is not more than 0.05%.
F-57 was recommended for Compound A for further pre-clinical development. The formulation supported a drug concentration at >2.5 mg/mL in emulsion.

### Example 6: Evaluation of Alternative Oil and Phospholipid in the F-57 Composition

F74-76 formulations were prepared containing Compound A (Form A), E-80 or soy lecithin, medium chain triglycerides (MCT) or Soybean Oil, Glycerin USP, edetate disodium di-hydrate USP (EDTA), NaOH (as pH adjustor), and sterile water for injection USP (SWFI), according to the compositions tabulated in Table 33.

TABLE 33

| | | F-74 | F-75 | F-76 |
|---|---|---|---|---|
| Formula (%) | F-57 | (200 g) | (200 g) | (200 g) |
| Compound A | 0.25 | 0.25 | 0.25 | 0.25 |
| E-80 | 10 | 10 | 0 | 0 |
| PL90G | 0 | 0 | 10 | 10 |
| MCT | 5 | 0 | 5 | 0 |
| Soybean Oil | 0 | 5 | 0 | 5 |
| Glycerin | 2.25 | 2.25 | 2.25 | 2.25 |
| EDTA | 0.0055 | 0.0055 | 0.0055 | 0.0055 |
| SWFI (q.s.) | 100 | 100 | 100 | 100 |

Procedures:
90% required lecithin, glycerin, EDTA and 30% of the required SWFI were added in a 250 mL primary container.
Mixed (high-shear) until a uniform coarse emulsion.
10% required lecithin, API and the oil per composition were added in a separate (50 mL) container. Mixed until completely dissolved API in oil phase at <65° C.
Added oil phase into the primary container. Mixed using high shear until uniform coarse emulsion obtained.
Adjusted pH by NaOH to 8.0-8.5 and bring with SWFI to q.s. to the batch weight (200 g).
The coarse emulsion was passed through a Microfluidizer (Registered Trademark) for 3 passes.
The emulsion was passed through 0.2 um filter.
Filled 5 mL in glass vials, stopper and crimp-seal.
Placed vials on stability at 2-8° C. and 40° C. for 4 weeks.
Tested for pH, appearance, HPLC assay/impurities, Z-Ave and % PFAT5. Results are shown in Table 34.

TABLE 34

| ID | | F-57 | F-74 | F-75 | F-76 |
|---|---|---|---|---|---|
| Bulk Appearance | | OWT | PPT | OWT | PPT |
| (Before Sterile Filtration) | | | | | |
| Post- | HPLC Assay | 2.51 | 1.90 | 2.50 | 2.18 |
| Sterile | (mg/mL) | | | | |
| Filtration | Appearance | OWT | OWT/PPT | OWT | OWT/PPT |
| | pH | 7.8 | 7.1 | 7.0 | 7.2 |
| | Z-Ave (nm) | 85 | 103 | 78 | 155 |
| | PFAT5 (%) | <0.001 | 0.004 | 0.001 | 0.004 |

PPT: Precipitation
OWYT: Off-white to Yellow Translucent Emulsion

Conclusion:
Only F-75 (containing PL90G/MCT) met the target Compound A concentration (2.5 mg/mL), in comparison with F-57.
F-74 (containing E-80/Soybean oil) and F-76 (containing PL90G/Soybean oil) did not support sufficient solubility and showed drug precipitation immediately after microfluidization preparation.

### Example 7: F-75 Stability Study

F-75 was placed at 2-8° C., 25° C. and 40° C. for 1, 2, and 3 Months to evaluate its stability in comparison with F-57. Results at time zero, 1 month, 2 months, and 3 months are shown in the tables below:

Time: zero

| | | | | | | | Pur- |
|---|---|---|---|---|---|---|---|
| Condi- | Appear- | | Z-Ave | PFAT5 | Assay | % Re- | ity |
| tion | ance | pH | (nm) | (%) | (mg/mL) | covery | (%) |
| 2-8° C. | OWYT | 7.0 | 78 | 0.001 | 2.50 | 100 | 100 |

Time: 1 Month

| | | | | | | | Pur- |
|---|---|---|---|---|---|---|---|
| Condi- | Appear- | | Z-Ave | PFAT5 | Assay | % Re- | ity |
| tion | ance | pH | (nm) | (%) | (mg/mL) | covery | (%) |
| 2-8° C. | OWYT | 6.9 | 79 | 0.037 | 2.48 | 99.3 | 100 |
| 25° C. | OWYT | 6.5 | 116 | 0.001 | 2.50 | 99.9 | 100 |
| 40° C. | OWYT | 6.4 | 200 | 0.006 | 2.50 | 99.7 | 100 |

Time: 2 Months

| | | | | | | | Pur- |
|---|---|---|---|---|---|---|---|
| Condi- | Appear- | | Z-Ave | PFAT5 | Assay | % Re- | ity |
| tion | ance | pH | (nm) | (%) | (mg/mL) | covery | (%) |
| 2-8° C. | OWYT | 7.2 | 84 | 0.001 | 2.45 | 98.2 | 100 |
| 25° C. | OWYT | 6.9 | 140 | 0.001 | 2.44 | 97.8 | 100 |
| 40° C. | OWO | 6.6 | 221 | 0.003 | 2.46 | 98.5 | 100 |

Time: 3 Months

| | | | | | | | Pur- |
|---|---|---|---|---|---|---|---|
| Condi- | Appear- | | Z-Ave | PFAT5 | Assay | % Re- | ity |
| tion | ance | pH | (nm) | (%) | (mg/mL) | covery | (%) |
| 2-8° C. | OWYT | 7.0 | 86 | 0.002 | 2.43 | 97.3 | 99.9 |
| 25° C. | OWYT | 6.6 | 158 | 0.002 | 2.49 | 99.5 | 99.8 |
| 40° C. | OWO, PS | n/a | n/a | n/a | n/a | n/a | n/a |

OWYT: Off-white to Yellow Translucent Emulsion

OWO: Off-white Opaque Emulsion

PS: phase separation

Conclusion:
F-75 remained stable after 3 Months at 2-8 and 25° C., and after 2 Months at 40° C.
Significant increase of mean droplet size (Z-Ave) was observed at 40° C. after 1 and 2 Mo, in comparison with F-57.
F-75 showed oil-phase separation after 3 Mo at 40° C.

### Example 8: Manufacture of a 2.5 mg/mL Emulsion

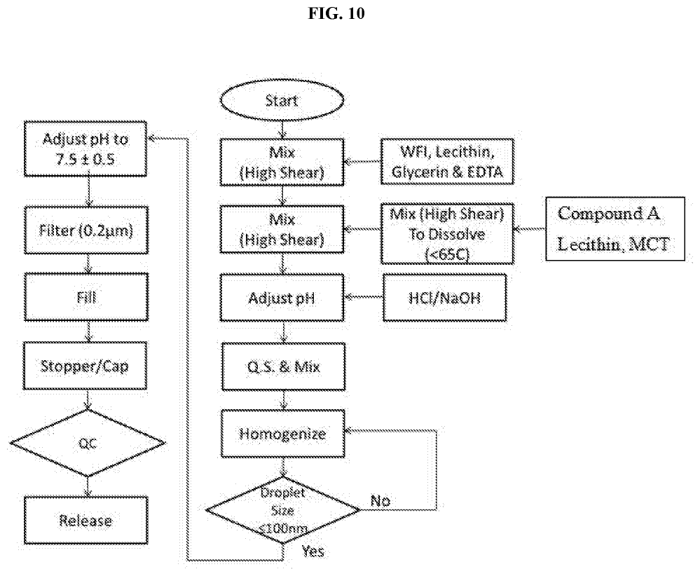

The Compound A (Form A) nanoemulsion was off-white to yellow translucent in appearance. The finished product was sterilized by 0.2 μm membrane filtration and has tonicity and pH near to physiological conditions. The product was filled in 100 mL USP Type I clear glass vials and stoppered with Flurotec stopper and crimp-sealed with Flip-Off overseal. Each mL of nanoemulsion contained 2.5 mg Compound A, 100 mg Egg Lecithin, 50 mg Medium-Chain Triglycerides (MCT) and 22.5 mg Glycerin, and 0.055 mg Edetate Disodium Dihydrate (EDTA-Na₂). The manufacturing process flowchart is outlined in FIG. 10. The preparation used a high-shear (rotor-stator) homogenizer to homogenize the crude emulsion and high-pressure Microfluidizer (Registered Trademark) to reduce average oil droplet size to not more than 100 nm. The order of addition and mixing steps (adding organic phase to aqueous phase) are unique to create a stable coarse emulsion. The composition and functionality are tabulated in Table 35.

TABLE 35

| | Percent | |
|---|---|---|
| Composition | (%, w/w) | Functionality |
| Compound A | 0.25 | Active |
| Egg Lecithin (E-80) | 10 | Emulsifier, Solubilizer |
| Medium-Chain Triglycerides | 5 | Solvent, Solubilizer |
| (MCT) USP | | |
| Glycerin USP | 2.25 | Tonicity Adjustor |
| Edetate Disodium, Dihydrate | 0.0055 | Chelating Agent |
| USP | | |
| 1N NaOH/HCl | pH adjustor | pH Adjustor |
| SWFI (q.s.) USP | 100 | Solvent |

### Example 9: Nanoemulsion Evaluation for Toxicity Studies

Large scale F-57 formulation (F57#0) as well as a vehicle formulation (Vehicle #0) (not comprising Compound A) were prepared. The composition of each formulation is tabulated in table 36.

TABLE 36

| | Vehicle#0 | F57#0 |
|---|---|---|
| Formula (%, w/w) | (4 Kg) | (2 Kg) |
| Compound A | 0 | 0.2 |
| Egg Lecithin (E-80) | 10 | 10 |
| Medium-Chain Triglycerides | 5 | 5 |
| (MCT) | | |
| Glycerin | 2.25 | 2.25 |
| EDTA-Na₂, Dihydrate | 0.0055 | 0.0055 |
| 1N NaOH/HCl | Adjust pH to 7-8 | Adjust pH to 7-8 |
| SWFI (q.s.) | 100 | 100 |

The stability of F-57 formulation (F57#0), vehicle formulation (Vehicle #0) at time 0 and at 6 Mo, and diluted formulations are shown in tables below.

Time zero (2-8° C.):

| | | | Mean | | | | |
|---|---|---|---|---|---|---|---|
| | | | Droplet Size | PFAT5 | Assay | % | Purity |
| Lot No. | Appearance | pH | (nm) | (%) | (mg/mL) | Target | (%) |
| Vehicle#0 | OWYT | 7.1 | 69 | 0.002 | 0.00 | n/a | n/a |
| F57#0 | OWYT | 7.9 | 70 | 0.002 | 2.01 | 100.5 | 100 |

OWYT: Off-white to Yellow Translucent Emulsion
n/a: Not applicable

6 Mo Stability (2-8° C.):

| | | | Mean | | | | |
|---|---|---|---|---|---|---|---|
| | | | Droplet | | | | Pur- |
| | Appear- | | Size | PFAT5 | Assay | % | ity |
| Lot No. | ance | pH | (nm) | (%) | (mg/mL) | Target | (%) |
| Vehicle#0 | OWYT | 7.4 | 66 | 0.002 | N/D | n/a | n/a |
| F57#0 | OWYT | 6.6 | 65 | 0.001 | 1.99 | 99.5 | 100 |

OWYT: Off-white to Yellow Translucent Emulsion
N/D: Not detectable
n/a: Not applicable

Diluted Emulsion Stability:

| | | | | Mean | | | | |
|---|---|---|---|---|---|---|---|---|
| | | | | Droplet Size | PFAT5 | Assay | % | Purity |
| ID | Condition | Appearance | pH | (nm) | (%) | (mg/mL) | Target | (%) |
| 0.0 mg/mL | After 24 h | OWYT | n/a | n/a | n/a | 0.00 | n/a | n/a |
| | at 2-8° C. | | | | | | | |
| 0.3 mg/mL | After 24 h | OWYT | n/a | n/a | n/a | 0.30 | 100 | 100 |
| | at 2-8° C. | | | | | | | |
| 0.8 mg/mL | After 24 h | OWYT | n/a | n/a | n/a | 0.81 | 101 | 100 |
| | at 2-8° C. | | | | | | | |
| 2.0 mg/mL | After 24 h | OWYT | n/a | n/a | n/a | 2.03 | 102 | 100 |
| | at 2-8° C. | | | | | | | |
| 0.0 mg/mL | After 8 h | OWYT | 7.1 | 69 | 0.002 | 0.00 | n/a | n/a |
| | at 25° C. | | | | | | | |
| 0.3 mg/mL | After 8 h | OWYT | 7.4 | 68 | 0.003 | 0.31 | 103 | 100 |
| | at 25° C. | | | | | | | |
| 0.8 mg/mL | After 8 h | OWYT | 7.5 | 69 | 0.003 | 0.82 | 103 | 100 |
| | at 25° C. | | | | | | | |
| 2.0 mg/mL | After 8 h | OWYT | 7.9 | 70 | 0.002 | 2.06 | 103 | 100 |
| | at 25° C. | | | | | | | |

### Conclusion:

The diluted emulsions were stable at room temperature after 8 h and 2-8° C. after 24 h. Lots Vehicle #0 and F57#0 (2 mg/mL) remained stable after 6 Mo at 2-8° C.

### Example 10: 3 Mo Stability Studies for Formulation F57 Emulsion

Large scale F-57 formulation (F57#1) as well as a vehicle formulation (Vehicle #1) were prepared. The composition of each formulation is tabulated in table 37.

TABLE 37

| | Vehicle#1 | F57#1 |
|---|---|---|
| Formula (%, w/w) | (18 Kg) | (18 Kg) |
| Compound A | 0 | 0.25 |
| Egg Lecithin (E-80) | 10 | 10 |
| Medium-Chain Triglycerides | 5 | 5 |
| (MCT) | | |
| Glycerin | 2.25 | 2.25 |
| EDTA-Na₂, Dihydrate | 0.0055 | 0.0055 |
| 1N NaOH/HCl | Adjust pH to 7-8 | Adjust pH to 7-8 |
| SWFI (q.s.) | 100 | 100 |

Microfluidization In-Process Data

| | Mean Droplet Size, | | | | | | | | |
|---|---|---|---|---|---|---|---|---|---|
| | Z-Ave (nm) | | | % Cutoff at 220 nm | | | pH | | |
| | 1^{st} | 2^{nd} | 3^{rd} | 1^{st} | 2^{nd} | 3^{rd} | 1^{st} | 2^{nd} | 3^{rd} |
| Lot No. | Pass | Pass | Pass | Pass | Pass | Pass | Pass | Pass | Pass |
| Ve- | 103 | 77 | 67 | 4.0 | 0.5 | 0.9 | 7.7 | 7.7 | 7.7 |
| hicle#1 | | | | | | | | | |
| F57#1 | 112 | 91 | 81 | 4.1 | 1.1 | 0.6 | 8.1 | 8.1 | 8.1 |

Lot Release Data

| | | | | Z-Ave | PFAT5 | Assay | % | Purity |
|---|---|---|---|---|---|---|---|---|
| Lot No. | App. | Density | pH | (nm) | (%) | (mg/mL) | Target | (%) |
| Vehicle#1 | OWYT | 1.01 | 7.1 | 70 | <0.001 | N/D | n/a | n/a |
| F57#1 | OWYT | 1.01 | 7.6 | 82 | 0.004 | 2.56 | 102.4 | 99.7 |

OWYT: Off-white to Yellow Translucent Emulsion
Z-Ave: Mean droplet size
n/a: Not applicable or not determined
N/D: Not detectable

Supplemental Size Distribution Data:

| | | Z-Ave | | | | | % Cutoff |
|---|---|---|---|---|---|---|---|
| Lot No. | Condition | (nm) | D_{(0.1, v)} | D_{(0.5, v)} | D_{(0.9, v)} | PDI | at 220 nm |
| Vehicle#1 | Lot Release | 70 | 24 | 41 | 86 | 0.174 | 0.1 |
| F57#1 | Lot Release | 82 | 25 | 45 | 97 | 0.198 | 0.5 |

Stability Data

| | | Osmolarity |
|---|---|---|
| Lot No. | Storage Condition | (mOsm/Kg) |
| Vehicle#1 | 2-8° C., 3 Mo | 365 |
| F57#1 | 2-8° C., 3 Mo | 367 |

The stability of F57#1 and its vehicle (Vehicle #1) were evaluated. The data are tabulated in Tables below:

Time Zero Emulsion Stability

| | | | | Z-Ave | PFAT5 | Assay | % | Purity |
|---|---|---|---|---|---|---|---|---|
| Lot No. | T | App. | pH | (nm) | (%) | (mg/mL) | Recovery | (%) |
| Vehicle#1 | 2- | OWYT | 7.1 | 70 | <0.001 | N/D | n/a | n/a |
| | 8° C. | | | | | | | |
| F57#1 | 2- | OWYT | 7.6 | 82 | 0.004 | 2.56 | 100.0 | 99.7 |
| | 8° C. | | | | | | | |

1 Mo Emulsion Stability

| | | | | Z-Ave | PFAT5 | Assay | % | Purity |
|---|---|---|---|---|---|---|---|---|
| Lot No. | T | App. | pH | (nm) | (%) | (mg/mL) | Recovery | (%) |
| Vehicle#1 | 2- | OWYT | n/a | n/a | 0.001 | N/D | n/a | n/a |
| | 8° C. | | | | | | | |
| F57#1 | 2- | OWYT | n/a | n/a | 0.001 | 2.57 | 99.2 | 99.7 |
| | 8° C. | | | | | | | |
| Vehicle#1 | 25° C. | OWYT | n/a | n/a | 0.001 | N/D | n/a | n/a |
| F57#1 | 25° C. | OWYT | n/a | n/a | <0.001 | 2.58 | 99.7 | 99.6 |
| Vehicle#1 | 30° C. | OWYT | n/a | n/a | 0.001 | N/D | n/a | n/a |
| F57#1 | 30° C. | OWYT | n/a | n/a | 0.002 | 2.57 | 99.5 | 99.6 |
| Vehicle#1 | 40° C. | OWYT | n/a | n/a | 0.001 | N/D | n/a | n/a |
| F57#1 | 40° C. | OWYT | n/a | n/a | 0.001 | 2.58 | 99.7 | 99.8 |

OWYT: Off-white to Yellow Translucent Emulsion
OWYO: Off-white to Yellow Opaque Emulsion
n/a: Not applicable or not determined
N/D: Not detectable
Z-Ave: Mean droplet size (nm)

2 Mo Emulsion Stability

| | | | | Z-Ave | PFAT5 | Assay | % | Purity |
|---|---|---|---|---|---|---|---|---|
| Lot No. | T | App. | pH | (nm) | (%) | (mg/mL) | Recovery | (%) |
| Vehicle#1 | 2- | OWYT | 6.4 | 72 | 0.002 | N/D | n/a | n/a |
| | 8° C. | | | | | | | |
| F57#1 | 2- | OWYT | 7.0 | 81 | 0.002 | 2.60 | 104.0 | 99.7 |
| | 8° C. | | | | | | | |
| Vehicle#1 | 25° C. | OWYT | 5.5 | 78 | 0.008 | N/D | n/a | n/a |
| F57#1 | 25° C. | OWYT | 6.2 | 84 | 0.004 | 2.58 | 103.2 | 99.7 |
| Vehicle#1 | 30° C. | OWYT | 4.9 | 114 | 0.005 | N/D | n/a | n/a |
| F57#1 | 30° C. | OWYT | 5.3 | 118 | 0.004 | 2.58 | 103.2 | 99.6 |
| Vehicle#1 | 40° C. | OWYT | 4.3 | 154 | 0.006 | N/D | n/a | n/a |
| F57#1 | 40° C. | OWYO | 4.6 | 149 | 0.001 | 2.59 | 103.6 | 99.6 |

OWYT: Off-white to Yellow Translucent Emulsion
OWYO: Off-white to Yellow Opaque Emulsion
n/a: Not applicable or not determined
N/D: Not detectable
Z-Ave: Mean droplet size (nm)

Supplemental Size Distribution Data (2 Month):

| | | Z-Ave | | | | | % Cutoff |
|---|---|---|---|---|---|---|---|
| Lot No. | T | (nm) | D_{(0.1, v)} | D_{(0.5, v)} | D_{(0.9, v)} | PDI | at 220 nm |
| Vehicle#1 | 2- | 72 | 31 | 47 | 87 | 0.168 | 0.2 |
| | 8° C. | | | | | | |
| F57#1 | 2- | 81 | 24 | 46 | 96 | 0.199 | 0.6 |
| | 8° C. | | | | | | |
| Vehicle#1 | 25° C. | 78 | 32 | 52 | 97 | 0.155 | 0.2 |
| F57#1 | 25° C. | 84 | 18 | 43 | 98 | 0.161 | 0.3 |
| Vehicle#1 | 30° C. | 114 | 42 | 78 | 184 | 0.163 | 5.9 |
| F57#1 | 30° C. | 118 | 43 | 74 | 232 | 0.245 | 10.3 |
| Vehicle#1 | 40° C. | 154 | 89 | 150 | 265 | 0.108 | 20 |
| F57#1 | 40° C. | 149 | 83 | 141 | 259 | 0.115 | 18 |

3 Mo Emulsion Stability

| | | | | Z-Ave | PFAT5 | Assay | % | Purity |
|---|---|---|---|---|---|---|---|---|
| Lot No. | T | App. | pH | (nm) | (%) | (mg/mL) | Recovery | (%) |
| Vehicle#1 | 2- | OWYT | 6.7 | 69 | 0.001 | N/D | n/a | n/a |
| | 8° C. | | | | | | | |
| F57#1 | 2- | OWYT | 7.0 | 81 | 0.001 | 2.63 | 102.8 | 100.0 |
| | 8° C. | | | | | | | |
| Vehicle#1 | 25° C. | OWYT | 5.6 | 77 | 0.002 | N/D | n/a | n/a |
| F57#1 | 25° C. | OWYT | 6.2 | 83 | 0.001 | 2.64 | 103.0 | 100.0 |
| Vehicle#1 | 30° C. | OWYO | 4.8 | 116 | 0.002 | N/D | n/a | n/a |
| F57#1 | 30° C. | OWYO | 5.3 | 105 | 0.002 | 2.63 | 102.8 | 100.0 |
| Vehicle#1 | 40° C. | OWYO | 4.1 | 153 | 0.001 | N/D | n/a | n/a |
| F57#1 | 40° C. | OWYO | 4.3 | 161 | 0.001 | 2.67 | 104.4 | 100.0 |

OWYT: Off-white to Yellow Translucent Emulsion
OWYO: Off-white to Yellow Opaque Emulsion
n/a: Not applicable or not determined
N/D: Not detectable
Z-Ave: Mean droplet size

Supplemental Size Distribution Data (3 Month):

| | | Z-Ave | | | | | % Cutoff |
|---|---|---|---|---|---|---|---|
| Lot No. | T | (nm) | D_{(0.1, v)} | D_{(0.5, v)} | D_{(0.9, v)} | PDI | at 220 nm |
| Vehicle#1 | 2- | 69 | 28 | 43 | 82 | 0.186 | 1.0 |
| | 8° C. | | | | | | |
| F57#1 | 2- | 81 | 29 | 46 | 99 | 0.194 | 0.6 |
| | 8° C. | | | | | | |
| Vehicle#1 | 25° C. | 77 | 35 | 55 | 99 | 0.141 | 0.2 |
| F57#1 | 25° C. | 83 | 34 | 55 | 107 | 0.174 | 1.1 |
| Vehicle#1 | 30° C. | 116 | 58 | 95 | 191 | 0.148 | 5.7 |
| F57#1 | 30° C. | 105 | 51 | 80 | 156 | 0.131 | 2.5 |
| Vehicle#1 | 40° C. | 153 | 87 | 147 | 266 | 0.111 | 20 |
| F57#1 | 40° C. | 161 | 90 | 161 | 304 | 0.141 | 28 |

8 Mo Emulsion Stability

| | | | | Z-Ave | PFAT5 | Assay | % | Purity |
|---|---|---|---|---|---|---|---|---|
| Lot No. | T | App. | pH | (nm) | (%) | (mg/mL) | Recovery | (%) |
| Vehicle#1 | 2- | OWYT | 6.1 | 70 | 0.001 | N/D | n/a | n/a |
| | 8° C. | | | | | | | |
| F57#1 | 2- | OWYT | 6.8 | 82 | 0.001 | 2.56 | 102.5 | 99.7 |
| | 8° C. | | | | | | | |
| Vehicle#1 | 25° C. | OWO | 4.7 | 93 | 0.001 | N/D | n/a | n/a |
| F57#1 | 25° C. | OWO | 5.1 | 89 | 0.001 | 2.50 | 97.7 | 99.8 |
| Vehicle#1 | 30° C. | OWO, PS | n/a | n/a | 0.392 | n/a | n/a | n/a |
| F57#1 | 30° C. | OWO | 4.4 | 141 | 0.009 | 2.53 | 99.0 | 99.7 |
| Vehicle#1 | 40° C. | OWO, PS | n/a | n/a | n/a | n/a | n/a | n/a |
| F57#1 | 40° C. | OWO, PS | n/a | n/a | n/a | n/a | n/a | n/a |

OWYT: Off-white to Yellow Translucent Emulsion
OWYO: Off-white to Yellow Opaque Emulsion
n/a: Not applicable or not determined
N/D: Not detectable
Z-Ave: Mean droplet size
PS: Phase separation

Supplemental Size Distribution Data (8 month):

| | | Z-Ave | | | | | % Cutoff |
|---|---|---|---|---|---|---|---|
| Lot No. | T | (nm) | D_{(0.1, v)} | D_{(0.5, v)} | D_{(0.9, v)} | PDI | at 220 nm |
| Vehicle#1 | 2- | 70 | 25 | 42 | 81 | 0.181 | 0.4 |
| | 8° C. | | | | | | |
| F57#1 | 2- | 82 | 21 | 37 | 92 | 0.200 | 0.5 |
| | 8° C. | | | | | | |
| Vehicle#1 | 25° C. | 93 | 43 | 68 | 129 | 0.148 | 0.9 |
| F57#1 | 25° C. | 89 | 37 | 61 | 118 | 0.150 | 0.6 |
| Vehicle#1 | 30° C. | n/a | n/a | n/a | n/a | n/a | n/a |
| F57#1 | 30° C. | 144 | 61 | 123 | 254 | 0.148 | 15.9 |

Conclusion:
F57#1 remained stable at 2-8° C., 25° C. and 30° C. after 8 Mo and at 40° C. after 3 Mo, meeting USP PFAT5 requirement (<0.05%). The % assay recovery by HPLC remains within 95-105% and purity >99%. Phase separation was observed at 40° C. after 8 Mo.
Vehicle #1 remained stable at 2-8° C. and 25° C. after 8 Mo and at 30 and 40° C. after 3 Mo meeting USP PFAT5 requirement (<0.05%). Phase separation was observed at 30 and 40° C. after 8 Mo.
A significant increase of Z-Ave (nm) from about 80 to 110 and 160 for F57#1 was observed at 30° C. and 40° C. after 3 Mo, respectively.
A significant increase of Z-Ave (nm) from about 70 to 120 and 150 for Vehicle #1 was observed at 30° C. and 40° C. after 3 Mo, respectively.
The appearance of all 2-8° C. stability samples remained unchanged, off-white to yellow translucent emulsion after 8 Mo. Their pH remains neutral (pH>6).
The appearance of all 30° C. and 40° C. stability samples turned slightly opaque after 3 Mo. A pH drop to ˜4 was observed for samples at 40° C. after 3 Mo.

### Example 11: Free-Fatty Acids (FFA), Peroxides Analysis

Vehicle formulation (Vehicle #2) and Compound A emulsion (F57#2) were prepared at 14-Kg scale. The composition is tabulated in table 38. The free-fatty Acids (FFA) and peroxides contents at 3 Mo and 6 Mo were analyzed and are shown in Table 39.

TABLE 38

| | Vehicle#2 | F57#2 |
|---|---|---|
| Formula (%, w/w) | (14 Kg) | (14 Kg) |
| Compound A | 0 | 2.5 |
| Egg Lecithin (E-80) | 10 | 10 |
| Medium-Chain Triglycerides, | 5 | 5 |
| Miglyol 812 (MCT) USP | | |
| Glycerin USP | 2.25 | 2.25 |
| EDTA-Na₂, Dihydrate, USP | 0.0055 | 0.0055 |
| 1N NaOH/HCl | Adjust pH to 7-8 | Adjust pH to 7-8 |
| SWFI, USP (q.s.) | 100 | 100 |

TABLE 39

| | | FFA | Peroxides | Zeta Potential |
|---|---|---|---|---|
| Lot No. | Conditions | (mmole/L)* | (ppm) | (mV) |
| Vehicle#2 | 2-8° C., 6 Mo | 2.1 | <0.5 | −26.4 |
| F57#2 | 2-8° C., 6 Mo | 2.0 | <0.5 | −23.3 |
| Vehicle#2 | 25° C., 3 Mo | n/a | <0.5 | n/a |
| F57#2 | 25° C., 3 Mo | 2.5 | <0.5 | n/a |
| Vehicle#2 | 25° C., 6 Mo | 3.0 | <0.5 | −27.3 |
| F57#2 | 25° C., 6 Mo | 3.2 | <0.5 | −25.2 |

*The FFA acceptance criteria is NMT 5 mmole/L in USP monograph for the marketed product Injectable Propofol Emulsion.

### Example 12: Analysis of Precipitate in 2.5 mg/mL Emulsion

In the later batches made, including one GMP batch (at 2.5 mg/mL), precipitation was detected after a shorter amount of time at 2-8° C.

Studies to determine the saturation solubility of Compound A (Form A) in the F57 vehicle were conducted. The precipitate in the GMP batch was collected and examined for crystalline structure and was found to be Form B.

It was speculated that the precipitation might be due to the following reasons:

1. Compound A was converted from Form A to a less soluble Form B in F57; and
2. Compound A concentration in F57 exceeded the solubility of Compound A in the F57 vehicle and the supersaturation led to a delayed precipitation. Precipitation times vary from 1 month to more than 1 year.

The term “solubility” used herein is defined as Compound A concentration where Compound A has reached a dissolution-precipitation equilibrium in F57 at a selected temperature. If the Compound A concentration in F57 is at or below the solubility, Compound A shall not precipitate. On the other hand, if Compound A concentration is higher than the solubility, Compound A is expected to precipitate over time.

To accurately determine Compound A solubility in F57, it was important to make sure that:
The dissolution-precipitation equilibrium was reached when the solubility was determined;
The equilibrium was reached in a practical amount of time (i.e. 1-2 months or less, instead of 1-2 yr);
The relationship between the solubility and the crystalline Form (A or B) was well understood.

To investigate the causes of precipitation and determine the Compound A solubility in F57 vehicle, the following seven (7) methods were applied to accurately determine solubility of Compound A in F57:

Method 1: Formulate Compound A in F57 at varied concentrations using GMP grade of Compound A and excipients with the regular process

Method 2: Formulate Compound A in F57 by introducing Compound A into a pre-formed F57 vehicle

Method 3: Observe Compound A in the previously made batches which already had extended incubation

Method 4: Conduct “top-down” and a “bottom-up” solubility studies in F57 vehicle

Method 5: Agitate Compound A GMP batch of F57 to promote the dissolution-precipitation equilibrium

Method 6: Add extra Form B seeds to Compound A GMP batch of F57 to promote Compound A crystal growth and precipitation

Method 7: Add Form B seeds to the samples made in method 1 to promote Compound A crystal growth and precipitation

Solubility Method and HPLC Method to Determine Compound A Concentration in F57

For solubility determination, a F57 sample (usually about 0.5 mL) was filtered through a 0.22 μm centrifuge filter (Costar Spin-X®+, P/N8169), the filtrate (free of any solid particle) was collected, diluted with isopropanol, and tested for Compound A concentration using the following HPLC method. Dissolution-precipitation equilibrium is reached once the measured filtrate concentration is constant, and that concentration can be regarded as the solubility.

| HPLC System | Agilent 1100 | | |
|---|---|---|---|
| Column | Agilent Technologies, Zorbax SB-C18, 4.6 × 150 | | |
| | mm, 3.5 μm (PN: 863953-902) plus SB-C18 4.6 × | | |
| | 12.5 mm Guard Column (PN: 820950-920) | | |
| Mobile Phase (MP) | MP A: 0.05% TFA in DI water* | | |
| | MP B: 0.05% TFA in methanol | | |
| | (*MP A is filtered through 0.8 μm nylon filters) | | |
| | Time | | |
| Gradient | (minutes) | % MP A | % MP B |
| | 0 | 60 | 40 |
| | 25 | 5 | 95 |
| | 27 | 0 | 100 |
| | 40 | 0 | 100 |
| | 40.5 | 60 | 40 |
| | 50 | 60 | 40 |

| | |
|---|---|
| Flow Rate | 1.0 mL/min |
| Detection | Ultraviolet (UV) 220 nm |
| Wavelength | |
| Column Temperature | 40° C. |
| Sample Temperature | 2-8° C. or ambient |
| Injection Volume | 10 μL |
| Run Time | 50 min |
| Diluent | 100% HPLC grade Isopropyl Alcohol (IPA) |
| Target Conc. | 0.25 mg/mL |

### Equilibrium Methods

Table 40 summarizes the general conditions used in the seven methods to promote the dissolution-precipitation equilibrium. Detailed procedures are described in each method section.

TABLE 40

| | | | | Method to |
|---|---|---|---|---|
| | | | | promote |
| | | | Initial | dissolution- |
| | | Method to add | concentration | precipitation |
| Method | Form | API into F57 | (mg/mL) | equilibrium |
| 1 | A | Regular process | 1.5, 2.0, 2.5 | None |
| | | | and 3.0 | |
| 2 | A | To a pre-formed | 1.0, 1.5, 2.0, | Seed with Form B |
| | | F57 vehicle | 2.5, 3.0 and 3.5 | API |
| 3 | A | Regular process | 2.5 | None |
| 4 | A and | To a pre-formed | 0 and 3.0 | Agitation |
| | B | F57 vehicle | | |
| 5 | A | Regular process | 2.5 | Agitation |
| 6 | A and | Regular process | 2.5 | Agitation and |
| | B | | | seed with Form B |
| | | | | API |
| 7 | A and | Regular process | 1.5, 2.0, 2.5 | Agitation and |
| | B | | and 3.0 | seed with Form B |
| | | | | API |

### Method 1

### Procedure:

4 batches (batch size: 1 L) of Compound A emulsion, containing Compound A at 1.5, 2.0, 2.5, and 3.0 mg/mL, respectively were prepared. The composition of each batch is according to the Table 41 below.

TABLE 41

| | Composition ID (%, w/w) | | | |
|---|---|---|---|---|
| | A1 | A2 | A3 | A4 |
| Compound A (GMP lot) | 0.15 | 0.20 | 0.25 | 0.30 |
| E-80 | 10 | 10 | 10 | 10 |
| MCT | 5 | 5 | 5 | 5 |
| Glycerin | 2.25 | 2.25 | 2.25 | 2.25 |
| NaOH/HCl | pH | pH | pH | pH |
| | adjustor | adjustor | adjustor | adjustor |
| EDTA | 0.0055 | 0.0055 | 0.0055 | 0.0055 |
| SWFI | QS to 100 | QS to 100 | QS to 100 | QS to 100 |

Compounded and processed aqueous phase, oil phase, and coarse emulsion according to the GMP batch process.
Verified and ensured complete drug dissolution in the oil phase and final coarse emulsion (visually and by microscopy). Recorded critical process parameters.
Transferred 100 mL each of final coarse emulsion into containers and store at 2-8° C. and 25° C. for appearance and microscopy evaluation after 24 h and 48 h, respectively.
Processed the remaining 800 mL coarse emulsion through Microfluidizer (Registered Trademark) to reach the average droplet size NMT 100 nm.
Passed each MF-processed emulsion through 0.22 μm filter and fill 50 mL in Type-I 100cc glass vials, stopper, and crimp-seal, similar to GMP process.
Placed sufficient vials at 2-8° C. and 25° C. for stability study (7 vials at each condition).
Pulled stability vials at 0, 1, 2, and 4 weeks to test for appearance, microscopy, pH, and concentration.
Used supernatant of emulsion sample for HPLC test, in the case of drug precipitation in the vials.

### Results:

All samples were visually clear after 4 weeks' storage at both 2-8° C. and 25° C., and remained at the same pH value. The concentration of each sample is listed in the table 42. Given that the GMP batch showed crystal precipitation after 1 month, this result indicated that the precipitation was more likely a random process. Seeding was applied to all samples to trigger and accelerate the precipitation process.

TABLE 42

| | Sample» | | | | | | | |
|---|---|---|---|---|---|---|---|---|
| | A1 | | A2 | | A3 | | A4 | |
| | 2-8° | 25° | 2-8° | 25° | 2-8° | 25° | 2-8° | 25° |
| | C. | C. | C. | C. | C. | C. | C. | C. |
| Time 0 | 1.53 | 1.53 | 2.03 | 2.03 | 2.52 | 2.52 | 3.03 | 3.03 |
| 1 wk | 1.55 | 1.55 | 2.06 | 2.04 | 2.56 | 2.54 | 3.03 | 3.06 |
| 2 wk | 1.56 | 1.56 | 2.07 | 2.06 | 2.58 | 2.54 | 3.06 | 3.08 |
| 4 wk | 1.57 | 1.55 | 2.07 | 2.09 | 2.56 | 2.59 | 3.07 | 3.09 |

### Method 2

### Procedure:

Prepared 6 emulsion (1 g each), containing Compound A (Form A) each at approximately 1.0, 1.5, 2.0, 2.5, 3.0, and 3.5 mg/mL, respectively, mixing Compound A and pre-formed F57 vehicle.
Weighed out Compound A (Form A) and F57 vehicle in polypropylene vials.
Mixed (high speed beadbeater, 600 sec) to achieve complete drug dissolution or saturation.
Placed the samples (which are without 0.2 μm filtration) at 2-8° C.
Tested at 0 and after 48 hr for appearance and microscopy. Recorded results.
If no sign of precipitation in any sample after 1 week, seeded each sample with 1-2 mg of Compound A (Form B) crystal.
Gently mixed to disperse the crystal in each sample.
Continued to store samples at 2-8° C. (All samples should contain crystals at this stage).
Tested 0.5 mL of supernatant of each 2-8° C. sample and passed through Spin-X 0.2 m for HPLC assay after 1, 2, and 5 weeks.

### Results:

All samples were visually clear 1 week after preparation. As shown in the table 43, after seeding with Compound A form B crystal, the samples with concentrations greater than 2.0 mg/mL started to decrease in API concentration, and reached a plateau (1.82-1.93 mg/mL) after two weeks. The samples (B1 and B2) that started with lower concentrations than 1.5 mg/ml slowly increased their API concentration. These results suggested the Compound A API dissolution-precipitation equilibrium in F57 is between 2.0 and 1.5 mg/mL. Therefore, the previous batches that contained API at 2.5 mg/L were supersaturated.

TABLE 43

| mg/mL | B1 | B2 | B3 | B4 | B5 | B6 |
|---|---|---|---|---|---|---|
| Initial conc. | 1.0 | 1.5 | 2.0 | 2.5 | 3.0 | 3.5 |
| 1 wk after seeding | 1.14 | 1.33 | 1.86 | 2.07 | 2.06 | 2.03 |
| 2 wk after seeding | 1.19 | 1.35 | 1.86 | 1.97 | 1.93 | 1.88 |
| 5 wk after seeding | 1.50 | 1.44 | 1.82 | 1.89 | 1.93 | 1.93 |

### Method 3

### Procedure:

Sample from previously prepared batches, determine the concentration by HPLC.

### Results:

The concentration of API in previous batches (GMP Batch and F57#1) was determined and listed in table 44. The samples from same batch (F57#1) showed different solution stability. One bottle of sample was still clear and did not decrease in concentration at all. On the other hand, another bottle showed visual precipitation and the concentration dropped to 1.84 mg/mL. This results suggested the precipitation of API from supersaturated solution is an opportunistic process. However, the results cannot make a conclusion whether the API in those two batches reached the dissolution-precipitation equilibrium.

TABLE 44

| | GMP Batch at | F57#1 (exp. 1) at | F57#1 (exp. 2) at |
|---|---|---|---|
| Sample | 2-8° C. 5 mon | 2-8° C. 13 mon | 2-8° C. 13 mon |
| Conc. (mg/mL) | 2.26 | 2.54 | 1.84 |

### Method 4

### Procedure:

The top-down method used high-energy homogenization to dissolve a set amount of Compound A (Form A) in the F57 vehicle to achieve supersaturation, allowing precipitation to take place over time to reach a dissolution-precipitation equilibrium in F57. The solubility of Compound A in the F57 vehicle was then determined.

The bottom-up method used a gentle mixing to slowly dissolve Compound A (Form A) in the F57 vehicle to reach the dissolution-precipitation equilibrium in F57. The solubility of Compound A in the F57 vehicle was then determined.
Top-down method: Add form A and B API, each into a separate tube containing the F57 vehicle, then apply extensive energy to each tube by homogenizer (BB, 600 sec) to obtain a clear solution, and store each tube at 2-8° C.
Bottom-up method: Add form A and B API, each into a separate tube containing the F57 vehicle, then gently shake each tube on a platform shaker at 2-8° C.
Pull sample aliquots at 1 day, 2 days, 1 week and 4 weeks to test for appearance and concentration.

### Results:

In the “top-down” approach, form A and B crystals were dissolved into F57 vehicle at strength 2.69 and 3.00 mg/mL, respectively. After 4 weeks' storage at 2-8° C., the concentration of each remained the same, as shown in table 45, which indicated no precipitation occurred.

TABLE 45

| mg/mL | Form A | Form B |
|---|---|---|
| 1 day | 2.69 | 3.00 |
| 2 day | 2.65 | 2.94 |
| 1 wk | 2.68 | 2.97 |
| 4 wk | 2.64 | 2.97 |

In the “bottom-up” approach, without applying extensive energy, the API spontaneously dissolved into a emulsion Vehicle to reach equilibrium (table 46). Overall, form A crystal showed a faster dissolution rate than form B crystal. The solubility of both crystal forms can reach 1.8 mg/mL at 2-8° C. in 7 weeks. This result further confirmed that the API in the previous GMP batch was supersaturated.

TABLE 46

| | Form A | Form A | Form B | Form B |
|---|---|---|---|---|
| mg/mL | (2-8° C.) | (25° C.) | (2-8° C.) | (25° C.) |
| 1 day | 1.20 | 2.18 | 0.75 | 1.30 |
| 2 day | 1.53 | 2.24 | 1.14 | 1.73 |
| 1 wk | 1.70 | 2.19 | 1.63 | 2.15 |
| 4 wk | 1.71 | 2.17 | 1.66 | 2.04 |
| 7 wk | 1.85 | ND | 1.80 | ND |

### Method 5

### Procedure:

Shake the GMP batch vials on platform shaker at 2-8° C. and 25° C., respectively. Pulled sample aliquots at 0, 2, 5, 6, and 9 weeks to test for appearance, and concentration.

### Results:

Although the GMP batch showed precipitation one month after preparation, the concentration was still 2.26 mg/mL after 5 months. In order to find out the final dissolution-precipitation equilibrium state faster, agitation was applied to speed up the precipitation process, since agitation can increase the exposure of seed in the solution. As shown in table 47, the concentration of API in the F57 GMP batch decreased to 1.88 mg/mL within weeks and reached equilibrium after 5 weeks.

TABLE 47

| Sample | Concentration (mg/mL) |
|---|---|
| Time 0 before agitation | 2.26 |
| 2 wk with agitation | 1.95 |
| 5 wk with agitation | 1.84 |
| 6 wk with agitation | 1.85 |
| 9 wk with agitation | 1.88 |

### Method 6

### Procedure:

Aliquot the GMP batch to small glass vials, dope each with form B as seed, and shake the vials on platform at 2-8° C. and 25° C., respectively.

Pull sample aliquots at 0, 2, 5, 6, and 9 weeks to test for appearance, and concentration.

### Results:

Additional seeding of API into the F57 GMP batch showed results consistent with the agitation study. The data further confirmed that API solubility in F57 is within the range of 1.8-1.9 mg/mL at 2-8° C.

TABLE 48

| Sample | Concentration (mg/mL) |
|---|---|
| 2 wk | 1.90 |
| 5 wk | 1.80 |
| 6 wk | 1.72 |
| 9 wk | 1.86 |

### Method 7

### Procedure

Add form B crystal (1 mg to 1 mL) to A1-A4 (samples made in section 3.1), and shake the vials on platform shaker at 2-8° C.

Pull sample aliquots at 0, 2, 3, and 5 weeks to test for appearance, and concentration.

### Results

The samples prepared in method 1 were clear after 1 month at 2-8° C. Form B crystal was added into each to initiate and accelerate the precipitation process. The concentration of all samples decreased to 1.8-1.9 mg/mL in 2 weeks and stayed within that range for the remainder of the study (table 49).

TABLE 49

| Sample | A1 | A2 | A3 | A4 |
|---|---|---|---|---|
| T0, pre-seeded | 1.54 | 2.03 | 2.53 | 3.03 |
| 2 wk, seeded | 1.88 | 1.91 | 1.78 | 1.97 |
| 3 wk, seeded | 1.93 | 1.89 | 1.89 | 1.99 |
| 5 wk, seeded | 1.89 | 1.90 | 1.89 | 1.87 |

### Methods Summary

The general observation and findings by all 7 methods are summarized in the table 50, according to the detailed observation and discussion pertaining to each method.

TABLE 50

| Method # | Measured Solubility (mg/mL) | Remark |
|---|---|---|
| 1 | ND | Equilibrium not reached |
| 2 | 1.8-1.9 | Equilibrium reached |
| 3 | ND | Equilibrium not reached |
| 4 | 1.8 | Equilibrium reached |
| 5 | 1.8-1.9 | Equilibrium reached |
| 6 | 1.8-1.9 | Equilibrium reached |
| 7 | 1.8-1.9 | Equilibrium reached |

### Conclusion:

All methods indicated Compound A (Form A) solubility in F57 was in the range of 1.8-1.9 mg/mL at 2-8° C.
The precipitation of Compound A (Form A) from previous batches was due to supersaturation.
The precipitate was predominately in Form B.

### Example 13: Stability of a 1.6 mg/mL Emulsion

The Stability of a 1.6 mg/mL was assessed as shown in Tables 51 (T=0), 52A and 52B (T=1 Mo), and 53A and 53B (T=3 Mo).

TABLE 51
Release Batch

| Assay | | |
|---|---|---|
| (test method) | Specifications | Results |
| Appearance | Translucent, non-separated, | Conforms |
| | white to yellowish emulsion | |
| | essentially free of visible | |
| | particulates | |
| Identity | Retention time and UV | Conforms |
| | spectrum are consistent with | |
| | reference standard | |
| Assay | 80-120% label claim (1.6 | 108% label claim |
| | mg/mL) | |
| Related Impurities (area %) | Report all NLT 0.10% NMT | Related Impurities: 0.0% |
| Individual Impurities: | 1.0% | (0.04%) Individual Impurities: <LOQ |
| Total Related Impurities | NMT 4.0% | Total Related Impurities: 0.0% |
| | | (0.04%) |
| pH USP<791> | pH 5 to 9 | 8 (7.61) |
| Osmolarity USP<785> | 340-400 mOsm/L | 363 mOsm/L |
| Volume in Container | NLT label claim | Conforms (82.5 mL) |
| Mean Droplet Diameter | MDD: LT 0.5 p.m D10, D50 | MDD: Conforms - |
| (MDD) | and D90: Report results | (62 nm or 0.062 μm) |
| | | D10: 21 nm; D50: 31 nm; |
| | | D90: 48 nm |
| Fatty Acid Concentration in IE | Report Results | [FFA]ₘₑₐₙ- 14.6 ΓΓ1M |
| (FFA) | | |
| Percent of Fat Residing in | NMT 0.05% | Conforms |
| Globules Larger than 5 μm | | Run# 1: 0.00% (0.001%); |
| (PFAT5)** USP<729> | | Run# 2: 0.00% |
| | | (0.001%); Run# 3: 0.00% |
| | | (0.001%) |
| Bacterial Endotoxin USP <85> | 15 EU/mL | <1.00 EU/mL |
| Sterility Tests USP <71> | Sterile | Sterile |
| Particulate Matter | ≥10 μm | ≥10 μm, Conforms |
| USP <788> Method II: | NMT 3000 particles/container | Beginning: 62; Middle: 37; |
| (microscopy) | ≥25 μm | End: 66 ≥25 μm, Conforms |
| | NMT 300 particles/container | Beginning 5; Middle: 4; End: 6 |

TABLE 52A
T = 1 Mo (5 ± 3° C., Ambient RH)

| Assay | | |
|---|---|---|
| (test method) | Specifications | Results |
| Appearance | Translucent, non-separated, | Conforms |
| | white to yellowish emulsion | |
| | essentially free of visible | |
| | particulates | |
| Assay | 80-120% label claim | 106% label claim |
| | (1.6 mg/mL) | |
| Related Impurities (area | Report all NLT 0.1% | Related Impurities: 0.0% (0.04%) |
| %) | NMT 1.0% | Individual Impurities: <LOQ |
| Individual Impurities: | NMT 4.0% | Total Related Impurities: 0.0% |
| Total Related Impurities | | (0.04%) |
| pH USP<791> | pH 5 to 9 | 7 (7.36) |
| Mean Droplet Diameter | MDD: LT 0.5 μm | MDD: Conforms - |
| (MDD) | D10, D50 and D90: Report | (62 nm or 0.062 μm) |
| | results | D10: 21 nm; D50: 31 nm; D90: 48 |
| | | nm |
| Fatty Acid | Report Results | [FFA]ₘₑₐₙ −= 14.5 mM |
| Concentration in IE (FFA) | | |
| Percent of Fat Residing in | NMT 0.05% | Conforms) |
| Globules Larger than 5 μm | | Run# 1: 0.00% Run# 2: 0.00% |
| (PFATS) USP<729> | | (0.001%); Run# 3: 0.00% |
| | | (0.001%) |

TABLE 52B
T = 1 Mo (25 ± 3° C./60% RH)

| Assay | | |
|---|---|---|
| (test method) | Specifications | Assay (test method) |
| Appearance | Translucent, non-separated, | Conforms |
| | white to yellowish emulsion | |
| | essentially free of visible | |
| | particulates | |
| Assay | 80-120% label claim | 106% label claim |
| | (1.6 mg/mL) | |
| Related Impurities (area %) | Report all NLT 0.1% | Related Impurities: 0.0% |
| Individual Impurities: | NMT 1.0% | (0.04%) |
| Total Related Impurities | NMT 4.0% | Individual Impurities: <LOQ |
| | | Total Related Impurities: 0.0% |
| | | (0.04%) |
| pH USP<791> | pH 5 to 9 | 7 (6.69) |
| Mean Droplet Diameter | MDD: LT 0.5 μm | MDD: Conforms - |
| (MDD) | D10, D50 and D90: Report | (65 nm or 0.065 μm) |
| | results | D10: 22 nm; D50: 29 nm; |
| | | D90: 46 nm |
| Fatty Acid Concentration in | Report Results | [FFA]ₘₑₐₙ = 15 mM |
| IE (FFA) | | |
| Percent of Fat Residing in | NMT 0.05% | Conforms |
| Globules Larger than 5 μm | | Run# 1: 0.00% (0.002); |
| (PFATS) USP<729> | | Run# 2: 0.00% (0.002%); |
| | | Run# 3: 0.00% (0.002%) |

TABLE 52A
T = 3 Mo (5 ± 3° C., Ambient RH)

| Assay | | |
|---|---|---|
| (test method) | Specifications | Results |
| Appearance | Translucent, non-separated, | Conforms |
| | white to yellowish emulsion | |
| | essentially free of visible | |
| | particulates | |
| Assay | 80-120% label claim | 104% label claim |
| | (1.6 mg/mL) | |
| Related Impurities (area | Report all NLT 0.10% | Related Impurities: 0.0% (0.04%) |
| %) | NMT 1.0 | Individual Impurities: ≥LOQ |
| Individual Impurities: | NMT 4.0% | (0.05%) |
| Total Related Impurities | | Total Related Impurities: 0.0% |
| | | (0.00%) |
| pH USP<791> | pH 5 to 9 | 7 (7.13) |
| Mean Droplet Diameter | MDD: LT 0.5 gm | MDD: Conforms - |
| (MDD) | D10, D50 and D90: Report | (62.3 nm or 0.0623 μm) |
| | results | D10: 19.3 nm; D50: 31.7 nm; |
| | | D90: 57.0 nm |
| Free Fatty Acid | Report Results | [FFA]mean = 17.5 mM |
| Concentration in IE (FFA) | | |
| Percent of Fat Residing in | NMT 0.05% | Conforms |
| Globules Larger than 5 μm | | Run# 1: 0.00% (0.001%); Run# 2: |
| (PFAT5) USP<729> | | 0.00% (0.0008%); Run# 3: 0.00% |
| | | (0.001%) |

TABLE 53B
T = 3 Mo (25 ± 3° C./60% RH)

| Assay | | |
|---|---|---|
| (test method) | Specifications | Assay (test method) |
| Appearance | Translucent, non-separated, | Conforms |
| | white to yellowish emulsion | |
| | essentially free of visible | |
| | particulates | |
| Assay | 80-120% label claim | 104% label claim |
| | (1.6 mg/mL) | |
| Related Impurities (area | Report all | Related Impurities: 0.0% (0.04%) |
| %) | NLT 0.10% | Individual Impurities: ≥LOQ(0.05%) |
| Individual Impurities: | NMT 1.0% | Total Related Impurities: 0.0% |
| Total Related Impurities | NMT 4.0% | (0.00%) |
| pH USP<791> | pH 5 to 9 | 6 (5.84) |
| Mean Droplet Diameter | MDD: LT 0.5 μm | MDD: Conforms - |
| (MDD) | D10, D50 and D90: Report | (66.6 nm or 0.0666 μm) |
| | results | D10: 22.4 nm; D50: 29.9 nm; |
| | | D90: 40.6 nm |
| Free Fatty Acid | Report Results | [FFA]ₘₑₐₙ = 15.9 mM |
| Concentration in IE (FFA) | | |
| Percent of Fat Residing in | NMT 0.05% | Conforms |
| Globules Larger than 5 μm | | Run# 1: 0.00% (0.003%); Run# 2 |
| (PFAT5) USP<729> | | 0.00% (0.002); Run# 3: 0.00 (0.002) |

### Example 14: Nanosuspension Formulation

Polyvinylpyrrolidone (PVP) and sodium deoxycholate formulations with 5 different cryoprotectants: 10% sucrose, 2% sucrose+5% mannitol, 5% sucrose+5% mannitol, 10% trehalose, 2% trehalose+5% mannitol; were prepared and evaluated.

Procedure for 10% sucrose nanosuspension:
Milled Compound A (Form A) at 100 mg/mL in 1% PVP and 0.25% sodium deoxycholate
Diluted to 50 mg/mL with 20% sucrose (10% final sucrose concentration)
Filled 4 mL of 50 mg/mL suspension into 10-mL vials
Lyophilized at −36° C. and 100 mTorr to dryness
Determined drying loss by pre- and post-lyo vial weights (n=5) to determine the amount of WFI to use for reconstitution

The powder formulation were resuspended to 50 mg/mL based on solids content and allowed to remain at ambient temperature and serially diluted to 10 and 1 mg/mL using D5W. The formulations were tested: optical microscopy and particle-size distribution (5 hours and 1 day) and assay and related substances.

### Results:

PSD and OM: No discernable changes over 24 hours in any formulation.

| | | Assay | Compound A |
|---|---|---|---|
| | | Compound A | Related Substances |
| Formulation | | (% label claim) | (% label claim, RRT) |
| 100 mg/mL | No | 100.7% (n = 3, | BLQ RRT 0.94 |
| | cryoprotectant | RSD = 3.8) | 0.17% RRT 1.02 |
| 10 mg/mL | 10% sucrose | 99.7% | BLQ RRT 0.94 |
| | | | 0.16% RRT 1.02 |
| | 2% sucrose/5% | 105.6% | BLQ RRT 0.94 |
| | mannitol | | 0.18% RRT 1.02 |
| | 5% sucrose/5% | 105.1% | BLQ RRT 0.94 |
| | mannitol | | 0.18% RRT 1.02 |
| | 10% trehalose | 108.2% | BLQ RRT 0.94 |
| | | | 0.18% RRT 1.02 |
| | 2% trehalose/5% | 103.3% | BLQ RRT 0.94 |
| | mannitol | | 0.18% RRT 1.02 |
| 1 mg/mL | 10% sucrose | 109.9% | 0.19% RRT 1.02 |
| | 2% sucrose/5% | 111.6% | 0.20% RRT 1.02 |
| | mannitol | | |
| | 5% sucrose/5% | 109.4% | 0.18% RRT 1.02 |
| | mannitol | | |
| | 10% trehalose | 112.6% | BLQ RRT 0.94 |
| | | | 0.19% RRT 1.02 |
| | 2% trehalose/5% | 106.3% | 0.19% RRT 1.02 |
| | mannitol | | |

For 100 mg/mL and 10 mg/mL: LOD˜0.04% LC; LOQ˜0.10% LC
For 1 mg/mL: LOQ˜0.2% LC
RRT 0.94 and RRT 1.02 are present in the bulk API at equivalent levels

### 3 Mo at 5° C. Stability of the 10% Sucrose Nanosuspension

| Test Parameter | T0 Results | T3 M Results |
|---|---|---|
| Appearance | White cake | White cake |
| Reconstitution/ | Quickly resuspended | Quickly resuspended |
| Resuspendability | (<5 seconds), no | (<5 seconds), no |
| | observable | observable |
| | agglomerates | agglomerates |
| Compound A | 94.3% | 101.5% |
| Assay | | |
| Compound A | 0.22% (RRT 0.65) | 0.24% (RRT 0.65) |
| Related | BLQ (RRT 0.94) | BLQ (RRT 0.75) |
| Substances | 0.16% (RRT 1.02) | BLQ (RRT 0.94) |
| | | 0.17% (RRT 1.02) |
| Particle Size | Mean: 0.11 μm | Mean: 0.11 μm |
| Distribution | D10: 0.07 μm | D10: 0.07 μm |
| | D50: 0.10 μm | D50: 0.10 μm |
| | D90: 0.1 μm | D90: 0.15 μm |
| Karl Fisher | 4.1% | 3.4% |

BLQ: Below limit of quantitation (0.1%)
¹average of n = 2 tests

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A pharmaceutical composition comprising N-(5-(6-chloro-2,2-difluorobenzo[d][1,3]dioxol-5-yl)pyrazin-2-yl)-2-fluoro-6-methylbenzamide, or a pharmaceutically acceptable salt thereof and a pharmaceutically acceptable excipient.

2. The pharmaceutical composition of claim 1, wherein the pharmaceutical composition is formulated as a homogeneous liquid, an emulsion, a nanosuspension, or a powder for reconstitution.

3. The pharmaceutical composition of claim 1, wherein the pharmaceutical composition is suitable for injection.

4. The pharmaceutical composition of claim 1, wherein N-(5-(6-chloro-2,2-difluorobenzo[d][1,3]dioxol-5-yl)pyrazin-2-yl)-2-fluoro-6-methylbenzamide is present as a free base.

5. The pharmaceutical composition of claim 1, wherein N-(5-(6-chloro-2,2-difluorobenzo[d][1,3]dioxol-5-yl)pyrazin-2-yl)-2-fluoro-6-methylbenzamide, or a pharmaceutically acceptable salt thereof is crystalline.

6. The pharmaceutical composition of claim 5, wherein crystalline N-(5-(6-chloro-2,2-difluorobenzo[d][1,3]dioxol-5-yl)pyrazin-2-yl)-2-fluoro-6-methylbenzamide is crystalline Form A which has at least one of the following properties:
(a) an X-Ray powder diffraction (XRPD) pattern substantially the same as shown in FIG. 1;
(b) an X-ray powder diffraction (XRPD) pattern comprising characteristic peaks at about 13.8° 2-Theta, about 14.2° 2-Theta, about 16.8° 2-Theta, about 19.2° 2-Theta, about 19.7° 2-Theta, about 21.1° 2-Theta, about 22.5° 2-Theta, about 22.7° 2-Theta, about 26.5° 2-Theta, and about 27.5° 2-Theta;
(c) a DSC thermogram substantially similar to the one set forth in FIG. 2; or
(d) a DSC thermogram with an endotherm having a peak at about 156.6° C.

7. The pharmaceutical composition of claim 1, wherein the pharmaceutical composition is formulated as an emulsion.

8. The pharmaceutical composition of claim 1, wherein the emulsion is suitable for injection.

9. The pharmaceutical composition of claim 1, wherein the pharmaceutically acceptable excipient is selected from the group consisting of lecithin, soybean oil (SBO), Medium Chain Triglycerides (MCT), cholesterol, Vitamin E succinate (VES), sucrose, glycerin, EDTA-Na₂, and any combination thereof.

10. The pharmaceutical composition of claim 1 comprising:
(i) N-(5-(6-chloro-2,2-difluorobenzo[d][1,3]dioxol-5-yl)pyrazin-2-yl)-2-fluoro-6-methylbenzamide;
(ii) lecithin;
(iii) Medium Chain Triglycerides (MCT);
(iv) Glycerin; and
(v) Water.

11. The pharmaceutical composition of claim 10, wherein the N-(5-(6-chloro-2,2-difluorobenzo[d][1,3]dioxol-5-yl)pyrazin-2-yl)-2-fluoro-6-methylbenzamide is present at a concentration from about 0.1 mg/mL to about 4.0 mg/mL.

12. The pharmaceutical composition of claim 10, wherein the N-(5-(6-chloro-2,2-difluorobenzo[d][1,3]dioxol-5-yl)pyrazin-2-yl)-2-fluoro-6-methylbenzamide is present at a concentration of less than about 1.8 mg/mL.

13. The pharmaceutical composition of claim 11, wherein the N-(5-(6-chloro-2,2-difluorobenzo[d][1,3]dioxol-5-yl)pyrazin-2-yl)-2-fluoro-6-methylbenzamide is present at a concentration of about 1.6 mg/mL.

14. The pharmaceutical composition of claim 10, wherein the N-(5-(6-chloro-2,2-difluorobenzo[d][1,3]dioxol-5-yl)pyrazin-2-yl)-2-fluoro-6-methylbenzamide is present at a concentration from about 0.1% to about 1% (w/w).

15. The pharmaceutical composition of claim 14, wherein N-(5-(6-chloro-2,2-difluorobenzo[d][1,3]dioxol-5-yl)pyrazin-2-yl)-2-fluoro-6-methylbenzamide is present at a concentration from about 0.1% to about 0.3% (w/w).

16.-29. (canceled)

30. The pharmaceutical composition of claim 7 substantially free of N-(5-(6-chloro-2,2-difluorobenzo[d][1,3]dioxol-5-yl)pyrazin-2-yl)-2-fluoro-6-methylbenzamide crystalline Form B which has at least one of the following properties:
(a) an X-Ray powder diffraction (XRPD) pattern substantially the same as shown in FIG. 4;
(b) an X-ray powder diffraction (XRPD) pattern comprising characteristic peaks at about 14.2° 2-Theta, about 17.1° 2-Theta, about 21.5° 2-Theta, about 25.4° 2-Theta, about 26.5° 2-Theta, and about 26.9° 2-Theta;
(c) a DSC thermogram substantially similar to the one set forth in FIG. 5; or
(d) a DSC thermogram with an endotherm having a peak at about 54.3° C. and about 155.9° C.

31. The pharmaceutical composition of claim 30, wherein the pharmaceutical composition is stable at about 5±3° C. for at least 3 months.

32.-36. (canceled)

37. The pharmaceutical composition of claim 1,
wherein the pharmaceutical composition is formulated as a powder for reconstitution.

38. The pharmaceutical composition of claim 1, wherein the pharmaceutical composition is suitable for injection once reconstituted with an aqueous carrier.

39.-47. (canceled)

48. The pharmaceutical composition of claim 1 comprising:
(i) N-(5-(6-chloro-2,2-difluorobenzo[d][1,3]dioxol-5-yl)pyrazin-2-yl)-2-fluoro-6-methylbenzamide;
(ii) polyvinylpyrrolidone (PVP);
(iii) sodium deoxycholate; and
(iv) sucrose.

49.-71. (canceled)
